(19) United States
(12) Patent Application Publication
(10) Pub. No.: US 2007/0162989 A1
(43) Pub. Date: Jul. 12, 2007

(54) Methods and compositions for treating motor neuron associated disorders

(76) Inventor: Andreas Hugo Kottmann, Bronx, NY (US)

Correspondence Address: WilmerHale/Columbia University, 399 PARK AVENUE, NEW YORK, NY, 10022, US

(21) Appl. No.: 11/183,209

(22) Filed: Jul. 14, 2005

Related U.S. Application Data
(60) Provisional application No. 60/588,262, filed on Jul. 14, 2004.

(51) Int. Cl. A01K 67/027; C12Q 1/68; C07H 21/04; C12P 21/06; C12N 5/06; C07K 14/47

(52) U.S. Cl. 800/18; 435/6; 435/69.1; 435/354; 530/350; 536/23.2

(57) ABSTRACT

The present invention provides a non-human animal (e.g., a mouse) that contains a plurality of genetically modified somatic cells (e.g., a motor neuron) having an exon 8 related loss-of-function modification in all WFS1 alleles. The present invention also provides a non-human animal that contains a plurality of genetically modified somatic cells having an expression cassette containing a reporter gene functionally linked to a WFS1 expression regulatory nucleic acid sequence. In addition, the present invention provides a variety of genetically modified cells and WFS1-related nucleic acids and compositions (including pharmaceutical compositions) containing the same. Also provided are methods for treating or preventing a motor neuron associated disorder (e.g., ALS), methods for identifying a modulator of WFS1 expression, methods for identifying a mimic agent of WFS1, methods for identifying a modulator of WFS1 in a motor neuron and methods for validating an animal model for a motor neuron associated disorder (e.g., ALS).

### CROSS-REFERENCE TO RELATED APPLICATIONS

The present application claims the benefit of U.S. Provisional Application Ser. No. 60/588,262, filed Jul. 14, 2004, which is incorporated herein by reference thereto.

### BACKGROUND OF THE INVENTION

Motor neuron associated diseases are progressive, degenerative disorders that affect nerves in the upper and/or lower parts of the body. Some of the diseases are inherited, while others may be acquired. Common motor neuron associated disorders include amyotrophic lateral sclerosis (ALS), progressive muscular atrophy (PMA), progressive bulbar palsy (PBP), progressive lateral sclerosis (PLS), and post-polio syndrome. Motor neuron associated diseases can be categorized into three major subgroups called primary lateral sclerosis (PLS, only the upper motor neurons are affected), progressive muscular atrophy (PMA, only the lower motor neurons are affected), and ALS (both are affected). Clinical data show that the majority of diagnoses of motor neuron associated diseases being ALS, while PLS and PMA each account for about 5% of cases. The incidence of MND is approximately 1-5/per 100,000 people, and a male has a 20% higher rate of incidence than a female. Approximately 5,000-6,000 new cases are diagnosed in the U.S. every year.

The onset of symptoms in a human patient is usually between 40-70 years of age. The disorder is characterized by the progressive loss of voluntary muscle contraction because of the destruction of nerve cells in the brain and the spinal cord that are responsible for the stimulation of the voluntary muscles. While the initial symptoms may be subtle, the disease causes progressive, severe physical disability. Symptoms may include difficulty swallowing, limb weakness, slurred speech, impaired gait, facial weakness and muscle cramps. Respiration may be also affected in the later stages of these diseases.

Amyotrophic lateral sclerosis (ALS) is a multi-systems disorder in which progressive degeneration of select spinal and cranial motor neuron populations is a defining feature. ALS is also associated with a continuum of cognitive dysfunction ranging from mild (ALSci) to a progressive dementia of the fronto-temporal type (ALS-FTD) in a significant percentage of patients. For the vast majority of patients with ALS, the etiology of the disorder was unknown. The quest for the elucidation of the disease mechanism at the molecular level would be aided by the discovery of (an) independent biochemical marker(s), which may help to distinguish vulnerable from resistant motor neurons.

It is known that motor neurons with rapid burst related Ca²⁺ oscillations, such as Hypoglossus motor neurons, appear to be those most vulnerable to motor neuron degeneration during ALS. Motor neurons achieve the rapid decay of Ca²⁺ transients by a combination of low intracellular Ca²⁺ buffer capacity and dynamic Ca²⁺ sequestration into the ER, allowing also for a high degree of variability in discharge patterns. Hence, corruption of the intracellular Ca²⁺ sequestration machinery could lead to local Ca²⁺ concentrations sufficiently high to activate mitochondria-related apoptotic mechanisms and associated signal cascades. In another aspect, oxidative stress, which occurs, for example, in superoxide dismutase enzyme (SOD) knock out mice, has been implicated in many human pathological conditions, including motor neuron associated disorders, such as, ALS.

However, the quest for the elucidation of a disease mechanism at the molecular level for motor neuron associated disorders, however, remains elusive. There exists a pressing need for the isolation of target genes involved with ALS and motor neuron associated disorders, such that novel methods and pharmaceutical compositions may be developed to manage, treat and/or prevent such diseases.

### SUMMARY OF THE INVENTION

The present invention provides a non-human animal (e.g., a mouse) that contains a plurality of genetically modified somatic cells (e.g., motor neurons), where the plurality of genetically modified somatic cells contain a loss-of-function modification in at least one WFS1 allele. In an embodiment of the invention, at least one of the plurality of genetically modified cells is selected from the group consisting of a central nervous system neuron, a peripheral nervous system neuron, a stem cell and a pancreatic β cell.

In another embodiment, the loss-of-function modification is a complete loss-of function modification. In yet another embodiment, the loss-of function modification is an exon 8 related loss-of-function modification. In a specific embodiment, the exon 8 related loss-of-function modification may be a mutation of an exon 8 related nucleic acid sequence (including, e.g., exon 8 of WFS1 gene). The mutation may be a frame shift mutation, a nonsense mutation, a missense mutation, and a splice donor site mutation.

In another embodiment of the invention, the non-human animal comprises a plurality of genetically modified cells that are homozygous WFS1 loss-of-function cells. In one embodiment of the invention, the non-human animal displays no pancreatic phenotype until at least about six months of age. In another embodiment, the pancreatic phenotype comprises a change in glucose level from about 100 mg/ml to about 340 mg/ml. In another embodiment, the non-human animal may further contain at least one generically modified germ cell having an exon 8 related loss-of-function modification in at least one WFS1 allele. Also provided are offspring of the non-human animal and genetically modified somatic cells and germ cells obtained therefrom.

The present invention further provides a non-human animal that contains a plurality of genetically modified somatic cells, where the plurality of genetically modified somatic cells contains an expression cassette having a reporter gene functionally linked to a WFS1 expression regulatory nucleic acid sequence and where the WFS1 expression regulatory nucleic acid sequence regulates an expression of the reporter gene. In one embodiment, the reporter gene may contain a nucleic acid sequence encoding a fluorescent polypeptide. Also provided are an offspring of the non-human animal and a genetically modified somatic cell obtained therefrom.

In addition, the present invention provides a genetically modified cell that contains a loss-of-function modification in at least one WFS1 allele. The genetically modified cell may also be selected from the group consisting of an embryonic stem cell, a central nervous system neuron, a peripheral nervous system neuron, a stem cell, and a pancreatic β cell.

In one embodiment, the loss-of-function modification is a complete loss-of-function modification. In another embodiment, the loss-of-function modification is an exon 8 related loss-of-function modification. In still another embodiment, the genetically modified cell is a homozygous WFS1 loss-of-function cell.

The present invention further provides a genetically modified cell containing an expression cassette having a reporter gene functionally linked to a WFS1 expression regulatory nucleic acid sequence, where the WFS1 expression regulatory nucleic acid sequence regulates an expression of the reporter gene.

The present invention also provides a variety of WFS1 related nucleic acids and compositions, including pharmaceutical compositions. In one embodiment, the present invention provides an expression vector containing a reporter gene functionally linked to a WFS1 expression regulatory nucleic acid sequence, where the WFS1 expression regulatory nucleic acid sequence regulates an expression of the reporter gene. In another embodiment, the present invention provides an isolated nucleic acid having essentially a nucleic acid sequence of SEQ ID NO: 1; SEQ ID NO: 2; SEQ ID NO: 3; SEQ ID NO: 4; and SEQ ID NO: 5. The isolated nucleic acid may be an RNA or a DNA sequence. In a further embodiment, the invention provides the compliment of the isolated nucleic acid sequence. In another embodiment, the invention provides a functional fragment of the isolated nucleic acid sequence which comprises at least 19 nucleotides. The isolated nucleic acid may be a WFS1-related antisense nucleic acid (e.g., an antisense RNA or a nucleic acid encoding an antisense RNA) and an WFS1-related interference nucleic acid (e.g., an interfering RNA or a nucleic acid encoding an interfering RNA). In yet another embodiment, the present invention provides an isolated nucleic acid containing an interfering nucleic acid (e.g., an interfering RNA) for regulating WFS1 expression/function in a cell, such as, a motor neuron, e.g., a motor neuron in a spinal cord slice or in a subject.

Also provided is a method for identifying a modulator of WFS1 expression, including the step of: (a) contacting a cell with a candidate agent, where the cell contains an expression cassette having a reporter gene functionally linked to a WFS1 expression regulatory nucleic acid sequence, and where the WFS1 expression regulatory nucleic acid sequence regulates an expression of the reporter gene; and (b) determining an effect of the candidate agent on the reporter gene expression, where a change of the reporter gene expression may indicate that the candidate agent may be a modulator of WFS1 expression. The modulator of WFS1 may be a factor or agent including, but not necessarily limited to, a small molecule compound, a polypeptide, an intracellular antibody, a polysaccharide, a lipid and a nucleic acid.

Additionally, the present invention provides a method for identifying a modulator of WFS1 in a motor neuron, including the steps of: (a) contacting a motor neuron with a candidate agent; and (b) determining an effect of the candidate agent on WFS1 function, where a change of the WFS1 function may indicate that the candidate agent may be a modulator of WFS1 in a motor neuron. The modulator of WFS1 may be a factor or agent selected from the group including, but not necessarily limited to, a small molecule compound, a polypeptide, an intracellular antibody (e.g., a scFv), a polysaccharide, a lipid and a nucleic acid (e.g., a nucleic acid having a nucleic acid sequence encoding an antisense RNA or an interfering RNA). A number of WFS1-mediated events may be used as indicators of WFS1 function, such as WFS1-mediated apoptosis, WFS1-mediated calcium responses, and WFS1-mediated phenotypes (e.g., muscle wasting, denervation of diaphragm, respiratory arrhythmia or psychiatric symptoms).

The present invention further provides a method for identifying a WFS1 mimetic, including the steps of: (a) contacting a genetically modified cell with a candidate mimic agent, where the genetically modified cell contains a loss-of-function modification in at least one WFS1 allele; and (b) determining an effect of the candidate mimic agent on WFS1-mediated signal transduction, where at least a partial restoration of the WFS1-mediated signal transduction function may indicate that the candidate mimic agent is a WFS1 mimetic. In one embodiment, the agent includes small molecule compounds, polypeptides, intracellular antibodies (e.g., a scFv), polysaccharides, lipids and nucleic acids (e.g., a nucleic acid having a nucleic acid sequence encoding an antisense RNA or an interfering RNA).

The present invention also provides a method for treating or preventing a motor neuron associated disorder (e.g., ALS) in a subject, including administering to the subject a pharmaceutical composition in an amount effective to enhance WFS1 function in the subject. In one embodiment, the WFS1 function may be enhanced using at least one protocol selected from the group consisting of a transcription level enhancement, a translation level enhancement, a post-translation level enhancement, an in vivo enhancement and an ex vivo enhancement.

Also provided is a method for validating an animal model of a motor neuron associated disorder (e.g., ALS), including the steps of: (a) determining a plurality of phenotypes of, a morphology and/or physiology of at least one motor neuron in, a candidate genetically modified animal under a given condition; (b) comparing the plurality of phenotypes with a plurality of phenotypes of an WFS1 model animal under the given condition; and (c) comparing the morphology and/or physiology of the at least one motor neuron with a morphology and/or physiology of at least one motor neuron in the WFS1 model animal, where an essentially identical match between the plurality of phenotypes, the morphology and/or the physiology may indicate that the candidate generically modified animal may serve as a model animal for the motor neuron associated disorder.

Other features and advantages of the present invention will become apparent from the following detailed description. It should be understood, however, that the detailed description and the specific examples, while indicating the preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 depicts a scheme for drug target discovery and evaluation which is further elucidated in Example 1 infra.

FIG. 2 demonstrates that wfs1 is expressed in the anatomic substrate of ALS with fronto temporal dementia. (A, B) High resolution, chromogenic RNA in situ analysis with wfs1 antisense probe on fresh frozen, cryostat sections of 8 week old mice. (A) Documentation of wfs1 expression in cranial motor neurons. Motor neuron populations are identified by name and cranial nerve. Sensitivity to ALS is indicated in red. B) expression of wfs1 in the fronto-temporal brain. Brain nuclei whose dysfunction could conceivably take part in the manifestation of fronto temporal dementia are identified.

FIG. 3 shows expression of wfs1 protein in spinal cord motor neurons and in large-bodied neurons of the dorsal root ganglia revealed by a guinea pig anti sera raised against wfs1: (A,C,E) 100 fold enlargements, (B,D,F) 200 fold enlargement. (A,B) confocal images of immunohistochemical stainings of wfs1 expression. (C,D) confocal images of immunohistochemical stainings of isl1,2 expression which is a marker for dorsal root ganglia neurons and motor neurons. (E,F) overlay of confocal images demonstrating that wfs1 protein is present in the soma of somatic Motor neurons and large bodied dorsal root ganglia neurons. (E) solid arrow points to somatic motor neurons, which express wfs1, open arrow points to pre ganglionic motor neurons, which do not express wfs1. (F) solid arrow points to the soma of somatic motor neurons and open arrow points to soma of proprioceptive dorsal root ganglia neurons, both of which express wfs1.

FIG. 4 shows expression of wfs1 protein in dendrites spinal cord—motor neurons and in CA1 neurons of the hippocampus A afferents of the dorsal root ganglia revealed as revealed by RNA in situ and immunhistochemical analysis: (C, 20 fold enlargement, 8 week old male mouse) RNA in situ revealing wfs1 expression in CA1 neurons (black arrow head) but not in the dentate gyrus, CA2 and CA3 areas (open arrow head) of the Hippocampus. Black dashed line indicates boundary between CA1 and CA2 subregions of the Hippocampus. (A, B, C, E, F) immunohistochemical staining of cryostat sections of fresh frozen tissues derived from 8 week old male mouse. (E, 40 fold enlargement) wfs1 protein expression in the soma of CA1 neurons. Yellow dashed line indicates boundary between CA1 and CA2 subregions of the Hippocampus. (F, 100 fold enlargement) counterstaining with DAPI (blue), a DNA stain revealing cell nuclei, reveals that wfs1 (red) is present in the soma of CA1 neurons and in the dendrites of these cells that reach into the molecular layers of the Hippocampus (yellow arrowheads). (A, 100 fold enlargement) presence of wfs1 protein in the soma and dendrites of spinal cord motorneurons. (B, 100 fold enlargement) vesicular Glutamine Transporter (vGluT1) staining in ventral horns on same section as in A. vGluT1 is present in axonal endplates of proprioceptive dorsal root ganglia cells, that contact motor neuron soma or dendrites in the ventral horns of the spinal cord. (C, 600 fold enlargement, overlay of A and B) punctate vGluT1 staining is present juxtaposed to puncta stained for wfs1 suggesting that wfs1 is present in post synaptic densities opposite of axonal endplates from proprioceptive neurons.

FIG. 5 depicts the design of the systemic and conditional wfs1 loss-of-function alleles and the transgenic wfs1 over—expression allele. (A) relates the primary structure of wfs1 protein to the intron-exon architecture of the genomic locus of the gene wfs1. Note that 63% of the protein is encoded in exon 8 of the gene including all transmembrane segments of the protein (green boxes). The red box signifies the part of the coding sequence which is deleted in the “exon 8 related loss-of-function modification”. (B) depicts the design of the conditional wfs1 loss-of-function allele. LoxP denotes signal recognition sequences for the recombinase cre. Intervening DNA between the two loxP sites will be exised by cre activity. In the unrecombined case a biscistronic mRNA will be transcribed which encodes a functional, full length wfs1 protein followed by a IRES tauLacZ marker cassette. In the cre recombined case, exon 8 and the tauLacZ marker is deleted and instead a mRNA is produced which encodes the first 37% of the wfs1 protein followed by an IRES-EGFP marker cassettes. IRES=Internal ribosomal entry site, SA+ splice acceptor ensuring the production of a bicistronic mRNA after cre mediated DNA recombination. In the unrecombined case, the marker protein expression of tauLacZ will stain the soma and axon of all cells that express wfs1, after cre mediated recombination all wfs1 expressing cells will express the marker protein EGFP. Hence the gene dosage of wfs1 can be assessed at a single cell resolution in histological preparations by monitoring expression of the two marker proteins. (C) Depicts the transgenic wfs1 over expression allele: expression of the full length mouse cDNA encoding wfs1 is achieved under the control of the Hb9 promoter and enhancer transcriptional regulatory sequences. Protein translation of wfs1 occurs from a bicistronic mRNA which also encodes the marker protein dsRED. The marker protein labels all cells that express wfs1 from the transgenic allele. IRES=internal ribosomal entry site.

FIG. 6 depicts the progressive nature of the neurological phenotype observed in wfs1−/− mice. The appearance of the various aspects of the phenotype manifest in a sequential and stereotypic fashion.

FIG. 7 shows muscle and denervation phenotype in wfs1 −/− animals. (A) wfs1+/− cross section through diaphragm; (B) wfs1 −/− cross section through diaphragm, open arrow points to a disintegrating NMJ revealed by a-Bungarotoxin staining; (C) quantification of numbers of vacuolized motor units and muscle thickness of wfs1 +/−and wfs 1−/− animals.

FIG. 8 demonstrates the dispersion of NMJs in wfs1 −/− mice as exemplified in the diaphragm muscle. (A, B) Diaphragm whole mount preparations stained with Cy3 conjugated a-Bungarotoxin. (A) shows a narrow band of NMJs lined up at the equator of each muscle fiber in wt animals. (B) demonstrates that NMJs are dispersed over a wide area centered around the equator of each muscle fiber and the typical spatial restricted pattern of NMJ localization is lost in wfs1 −/− animals. The phenotype observed is reminiscent of the lack of clustering of NMJs observed in Hb9−/− mice.

FIG. 9 demonstrates partial denervation of the diaphragm muscle at 6 months of age in wfs1 −/− mice. (A, B) Diaphragm whole mount preparations stained with Cy3 conjugated a-Bungarotoxin and antisera against Neurofilament and Synaptophysin which are revealed with secondary antisera conjugated to FITC. (A) shows individual axons in green innervating NMJs in red in wfs1 +/−animals. (B) demonstrates that normal innervation of NMJs in wfs1−/− animals is disrupted caused by axon fragmentation leading to NMJ “ghosts” (white arrow) which exhibit normal a Bungarotoxin staining in the absence of innervation by axons. (C) quantification of the area of NMJs and numbers of NMJ “ghosts” in wfs1 +/−and wfs1 −/− animals: area and complexity of NMJs in wfs1 −/− animals is slightly reduced. The appearance of completely denervated NMJs (ghosts) in wfs1−/− is dramatically increased (P=0.0025)

FIG. 10 demonstrates swelling of somatic motor neurons exemplified with an analysis of motor neurons in the ventral horn of the spinal cord at level cervical 3 at 6 months of age. (A, B) 16 micrometer cryostate sections through ventral horns of spinal cords at 200 fold enlargement stained immunohistochemically for choline-acetyl transferase (ChAT, revealed by cy3 conjugated secondary antibodies) and wfs1 (revealed by FITC conjugated secondary antibodies). (B) reveals severe swelling of Motor neurons in the absence of wfs1. (c) overview for localization of sections in A,B within the spinal cord and quantification of motor neuron swelling in motor neurons in wfs1 −/− animals.

FIG. 11 depicts the nucleic acid sequence of the promoter WFS1 promoter fragment (SEQ ID NO: 1).

FIG. 12 depicts the nucleic acid sequence of the WFS1 enhancer 1 (5′ enhancer) fragment (SEQ ID NO: 2).

FIG. 13 depicts the nucleic acid sequence of the WFS1 enhancer 2 (intron 1 enhancer) fragment (SEQ ID NO: 3).

FIG. 14 depicts the nucleic acid sequence of the WFS1 enhancer 3 (intron 3 enhancer) fragment (SEQ ID NO: 4).

FIG. 15 depicts the nucleic acid sequence of the WFS1 enhancer 4 (intron 6 enhancer) fragment (SEQ ID NO: 5).

### DETAILED DESCRIPTION OF THE INVENTION

As used herein and in the appended claims, the singular forms “a,” “an” and “the” include plural references unless the content clearly dictates otherwise. Thus, for example, reference to “a cell” includes a plurality of such cells and equivalents thereof known to those skilled in the art, and reference to “the nucleic acid” is a reference to one or more nucleic acids and equivalents thereof known to those skilled in the art and so forth. All publications, patent applications, patents and other references mentioned herein are incorporated by reference in their entirety.

The present invention relates generally to systems, compositions and methods for identifying modulators/mimics of WFS1 function and/or WFS1-mediated signal transduction pathway. The present invention also relates to methods for treating and preventing motor neuron associated disorders, such as ALS. The present invention further provides compositions (e.g., pharmaceutical compositions) for treating and preventing such disorders, and methods of making and using the same.

It is known that mutations in the Wolfram Syndrome (WS) gene wolframin (WFS1) may cause progressive neurodegenerative disease WS. WS is an autosomal recessive disorder most frequently characterized by juvenile, non-immune system dependent, diabetes mellitus, progressive optic and brain stem atrophy. WS patients die from neurological complications with a median age of 30. A range of psychiatric conditions also have been associated with WS, including dementia, psychosis, affective disorder, major depressive disorder, suicide and assaultive behavior. Heterozygous carriers whose genome contains a wild-type WFS1 allele and a WFS1 allele having a loss-of-function mutation appear morphologically normal, but exhibit a 26 times higher lifetime susceptibility towards severe psychiatric complications.

In one aspect, the present invention provides a non-human animal containing a plurality of genetically modified somatic cells, which include loss-of-function modification in at least one WFS1 allele. The non-human animal may be any animal whose wild-type genome contains at least one copy of WFS1 gene or a functionally and structurally equivalent thereof including, without limitation, a domestic animal (e.g., a dog and a cat) or a commercial animal (e.g., a chicken, a fish, an insect, a mouse, a rat, a rabbit, a cow, a horse, a monkey and a chimpanzee). In a preferred embodiment, the non-human animal is a rodent, such as a mouse. Also provided is a progeny or offspring of a non-human animal of the present invention, which contains a plurality of genetically modified somatic cells whose genome includes a wild-type WFS1 allele and a null WFS1 allele containing loss-of-function modification.

As used herein, the term “a genetically modified cell” refers to a cell whose genome has been genetically manipulated by human activities including, without limitation, a cell containing artificially introduced exogenous nucleic acid and a cell whose genome contains artificially-produced mutations (e.g., a deletion mutation, a frame shift mutation, a nonsense mutation, a missense mutation and a splice donor site mutation). The term may also include a progeny of a genetically modified cell even though, under certain circumstances, the progeny may be of a different type as compared to the original genetically modified cell. For example, a genetically modified cell may be an embryonic stem cell while the progeny genetically modified cell may be a motor neuron. A genetically modified cell may be any somatic or germ cell including, without limitation, a central nervous system neuron, a peripheral nervous system neuron, a stem cell, and a pancreatic β cell. In one embodiment, a genetically modified somatic cell may be a motor neuron or a stem cell (e.g., an embryonic stem cell). In another embodiment, all live cells (including somatic cells and germ cells) in a non-human animal of the present invention contain loss-of-function modification in at least one WFS1 allele.

The genome of a wild-type cell may contain one, two or more WFS1 alleles. In one embodiment, all WFS1 alleles in a cell may be genetically silenced through at least one exon 8 related loss-of-function modification and, thereby, generating a homozygous WFS1 null cell. The genetic silencing may be partial or complete, e.g., through a partial or complete exon 8 related loss-of-function mutation. The genetic silencing effect may be permanent (e.g., a permanent deletion mutation) or temporary (e.g., a transient expression of an interfering RNA). The term, “exon 8 related loss-of-function modification” or “exon 8 related loss-of-function mutation,” as used herein, denotes a partial or complete loss-of-function mutation or genetic modification of the exon 8 of WFS1 gene. For example, an exon 8 related loss-of-function modification may be a partial or complete deletion of exon 8 from the genome of a cell, such as, by homologous recombination, or a point mutation. An exon 8 related loss-of-function modification may also be a loss-of-function mutation of a nucleic acid sequence essential to the function of exon 8, such as a mutation at a splice site, thereby preventing a processing of WFS1 mRNA to produce functional WFS1 polypeptide. As used herein, the term “homozygous WFS1 null cell” refers to a cell containing identical loss-of-function mutations in all WFS1 alleles, as well as a cell including different exon 8 related loss-of-function mutations so long as all WFS1 alleles contain exon 8 related loss-of-function mutations.

Due to the peculiar genomic structure of the gene encoding WFS1, 65% of the protein is encoded in a single exon, exon 8. Included in exon 8 are all transmembrane domains and the carboxyl terminal third of the protein. The cell physiological function of WFS1 is based on its localization in the ER membrane. A multitude of missense and nonsense mutations, among them deletios of just 1 amino acid, have been found in the protein portion encoded by exon 8 and cause wolfram syndrome in humans. Compound loss-of-function mutations are the most common cause of wolfram syndrome. Hence, the exon 8 related loss-of-function mutation described herein likely leads to a complete genetic ablation of WFS1 function.

The present invention also provides a non-human animal (e.g., a rodent such as a mouse) including a plurality of genetically modified somatic cells which may contain an expression cassette, where the expression cassette contains a reporter gene functionally linked to a WFS1 expression regulatory nucleic acid sequence and where the WFS1 expression regulatory nucleic acid sequence may regulate an expression of the reporter gene. Also provided is a progeny or offspring of a non-human animal of the present invention and genetically modified somatic cells obtained from a non-human animal and a progeny thereof.

An expression cassette in accordance with one embodiment of the present invention may be a nucleic acid containing a WFS1 expression regulatory nucleic acid sequence and a nucleic acid sequence encoding a reporter gene. A WFS1 expression regulatory nucleic acid sequence may include a nucleic acid sequence which regulates the spatial and/or temporal expression pattern of a WFS1 gene and which generally may locate in the 5′ UTR region, upstream of the first start codon. A WFS1 expression regulatory nucleic acid sequence may contain at least one functional subunit such as, without limitation, a promoter, an enhancer and a DNA polymerase binding site. In an embodiment, a WFS1 expression regulatory nucleic acid sequence includes a nucleic acid sequence having essentially a nucleic acid sequence of SEQ ID NO: 1; SEQ ID NO: 2; SEQ ID NO: 3; SEQ ID NO: 4; and SEQ ID NO: 5.

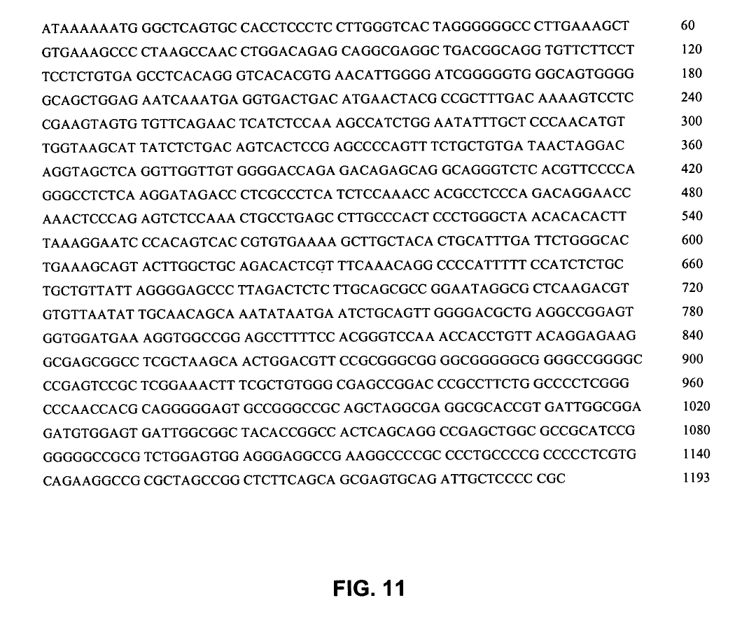

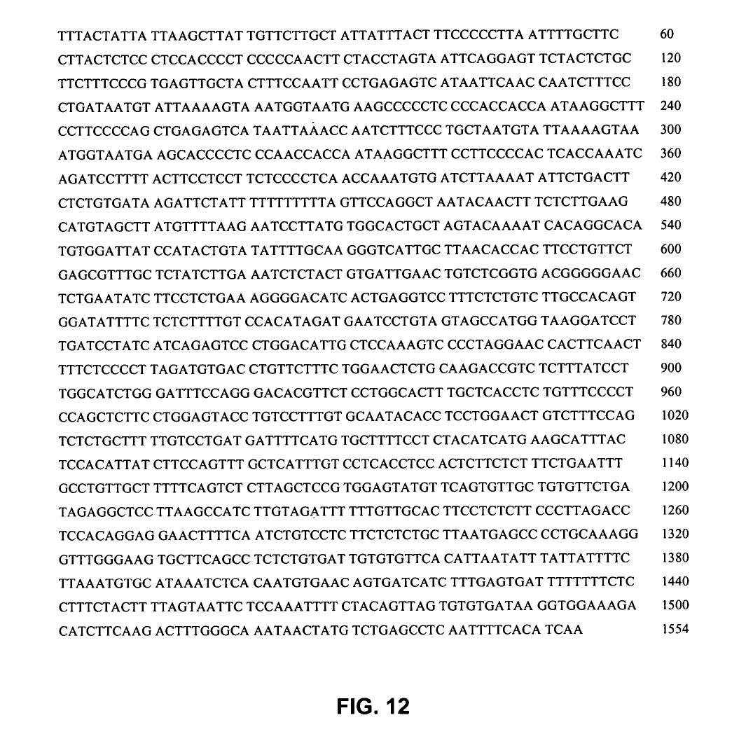

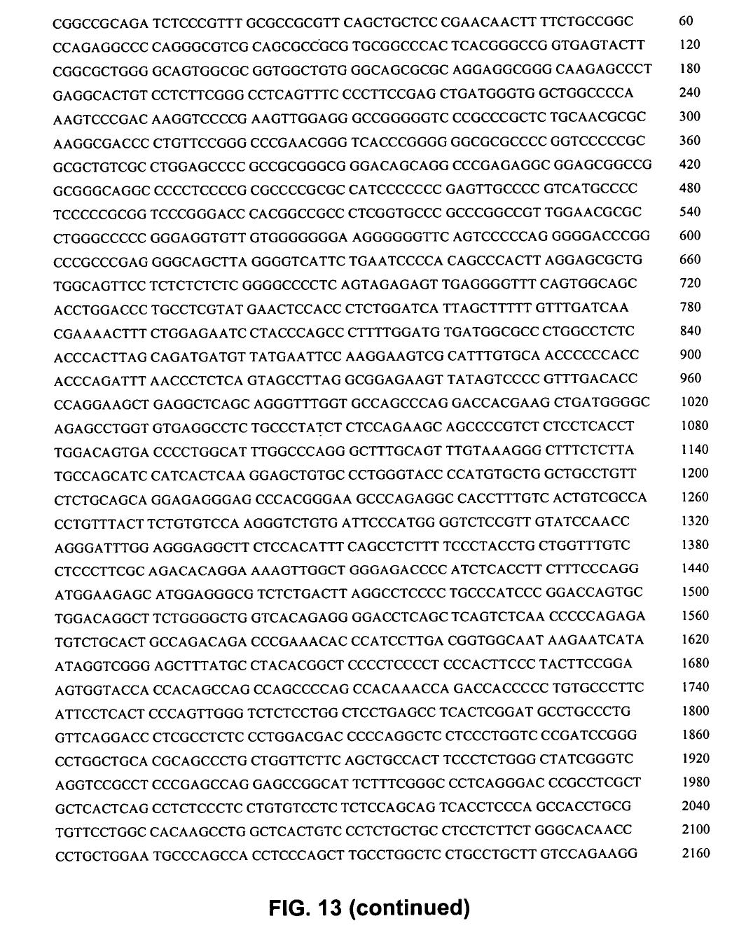

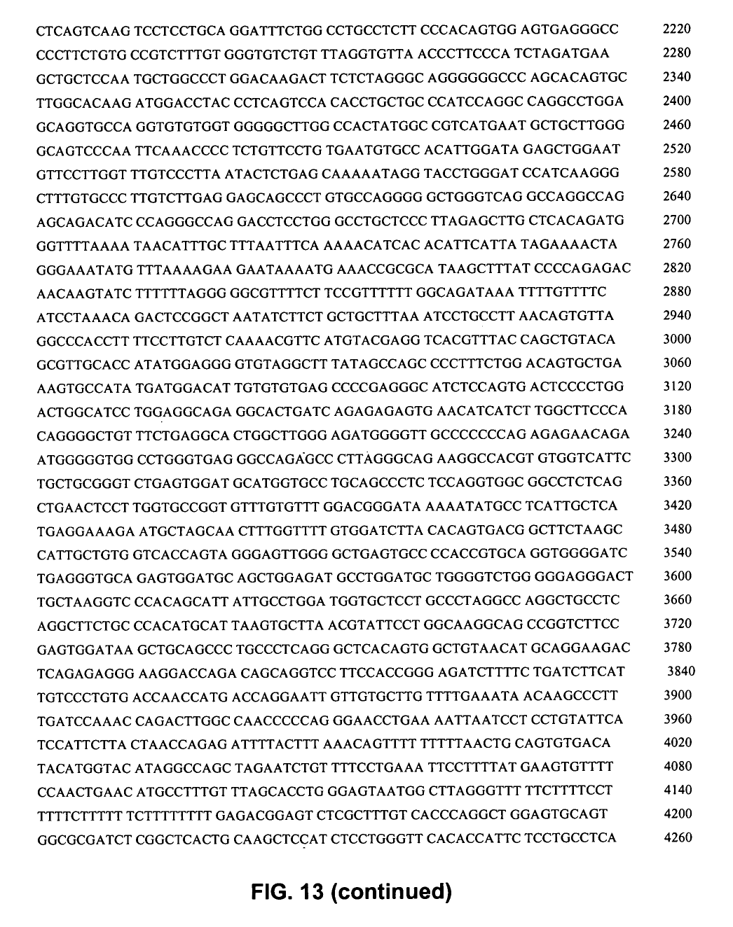

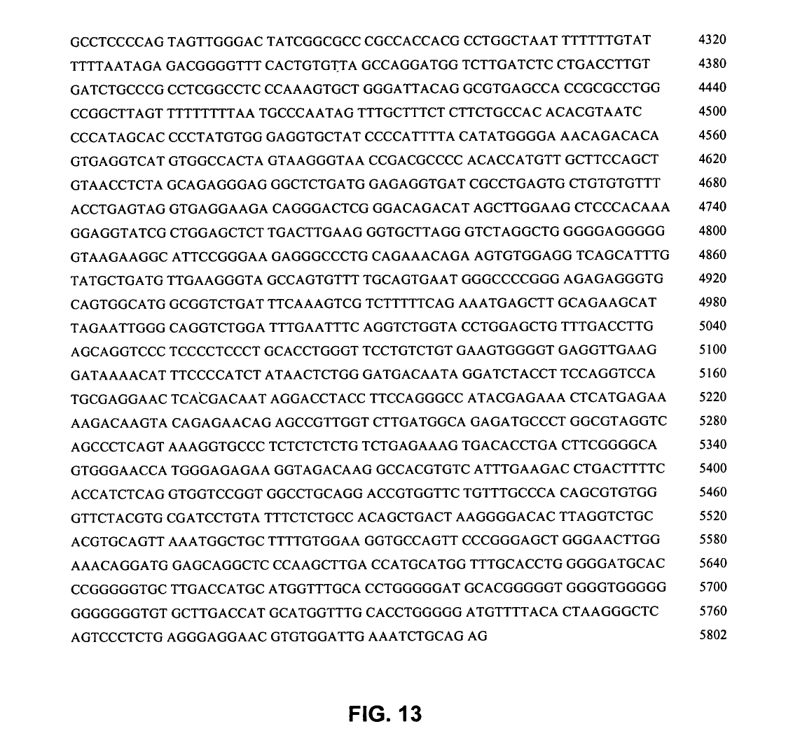

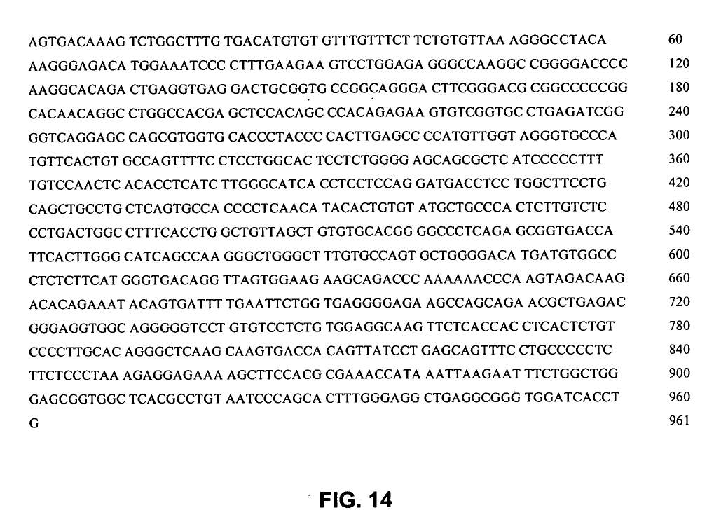

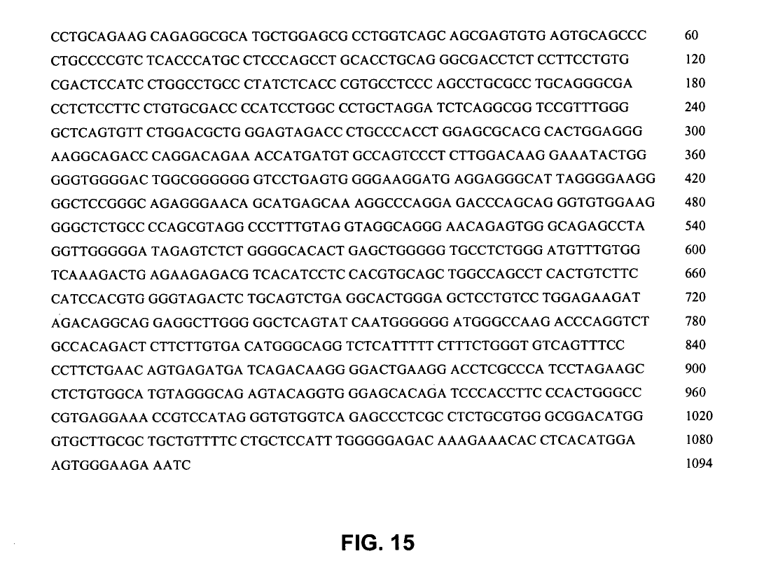

All regulatory sequences have been characterized using the human genomic WFS1 gene sequence (May 2004 assembly of human genome). The promoter fragment (FIG. 11; SEQ ID NO: 1) (chr4: 6,388,512-6,389,704) is a 1192 base pair fragment that includes exon 1, a non-coding exon and part of a CpG island. The enhancer 1 (5′ enhancer) sequence (FIG. 12; SEQ ID NO: 2) (chr4: 6,386,959-6,388,512 is a 1553 base pair fragment that is continuous with the 5′ end of the promoter fragment. The enhancer 2 (intron 1 enhancer) sequence (FIG. 13; SEQ ID NO: 3) (chr4: 6,389,704-6,395,505) is a 5801 base pair fragment that is continuous with the 3′ end of the promoter fragment. The enhancer 3 (intron 3 enhancer) sequence (FIG. 14; SEQ ID NO: 4) (chr4: 6,406,840-6,407,800) is a 960 base pair fragment that is discontinuous with the other sequences. The enhancer 4 (intron 6 enhancer) sequence (FIG. 15; SEQ ID NO: 5) (chr4: 6,411,750-6,412,843) is a 1093 base pair fragment that is also discontinuous with the other sequences.

The reporter gene in an expression cassette of the present invention may be functionally or operably linked to a WFS1 expression regulatory nucleic acid. Because the expression of the reporter gene is under control of the same expression regulation mechanism as that of a wild-type WFS1 gene, the expression of the reporter gene, or the lack of it, which may be readily detected and/or quantified, under a given condition (e.g., in a compound screening assay) may be used as an indicator of WFS1 expression under similar condition. Common reporter genes used in the art encode for example, without limitation, secreted alkaline phosphatase, β-galactosidase, β-glucoronidase, β-lactamase, catechol dehydrogenase, chloramphenicol acetyltransferase, green fluorescent protein, horseradish peroxidase, luciferase, nopaline synthase, octapine synthase and red fluorescent protein. In one embodiment, the reporter gene may encode a fluorescent polypeptide such as a green fluorescent polypeptide. In another embodiment of the present invention, the genetically modified somatic cells may further contain an exogenous tissue-specific expression-controlling element (e.g., a cre-lox system), where the exogenous tissue-specific expression-controlling element regulates an expression of the reporter gene.

The expression cassette may be introduced into a cell of a non-human animal of the present invention through an expression vector. An expression vector may be any vector suitable for carrying and expressing an exogenous nucleic acid in a cell, such as, without limitation, a cosmid, a plasmid, a virus and an artificial chromosome. A plethora of expression vectors is well known in the art and commercially available from, for instance, BD Biosciences Clontech, Invitrogen, Promega and Qiagen. Techniques for obtaining and/or incorporating a nucleic acid into an expression vector and subsequently introducing the vector into a cell, in vitro, in vivo or ex vivo, are well known in the art. In one embodiment, a vector carrying an expression cassette of the present invention may be introduced into an embryonic stem cell in vitro, which may develop into a transgenic animal. Depending on the type(s) of expression regulatory sequence(s) used, a transgene in the expression cassette/vector may be expressed universally in all the cells of the transgenic animal or in the case that a spatial or temporal promoter is used to drive the expression of the transgene, in certain type(s) of cells or in cells in certain developmental stage. As used herein, the term “transgene” refers to a nucleic acid molecule (e.g., a DNA, a RNA, a DNA/RNA hybrid, a gene or a fragment thereof) that has been/will be introduced into a cell or an animal by experimental manipulation, wherein the introduced gene is not endogenous to the animal or is a modified or mutated form of a gene that is endogenous to the animal (including, e.g., a wild-type copy of a target gene when the endogenous copy of the gene is a loss-of-function mutant). A modified or mutated form of an endogenous gene may be produced through human intervention (e.g., by introduction of a point mutation, introduction of a frame shift mutation, deletion of a portion or fragment of the endogenous gene, insertion of a selectable marker gene, insertion of a termination codon, etc.).

A transgenic non-human animal may be produced by any suitable methods known in the art including, without limitation, introduction of a transgene into an embryonic stem (“ES”) cell, newly-fertilized egg, or early embryo of a non-human animal; integration of a transgene into a chromosome of the somatic and/or germ cells of a non-human animal; and any methods described herein. In one embodiment of the present invention, the transgenic non-human animal is produced by introduction of a transgene into an ES cell.

By way of example, a transgenic non-human animal (e.g., a mouse line) of the present invention may be established by pronucleus injection of a construct that uses a fragment (e.g., ˜9 kB) comprising the 5′ upstream region of the murine HB9 gene (Arber, et al., Requirement for the homeobox gene Hb9 in the consolidation of motor neuron identity. Neuron, 23:659-764, 1999), followed by a 5′ splice substrate (Choi, et al., A generic intron increases gene expression in transgenic mice. Mol. Cell. Biol., 11:3070-74, 1991), an expression cassette of the present invention and a bovine growth hormone polyadenylation signal. ES cell lines then may be derived from heterozygous blastocysts, as previously described (Abbondanzo, et al., Derivation of embryonic stem cell lines. Methods Enzymol., 225:803-23, 1993). A genetic line may be selected based upon its capacity to integrate into developing mouse blastocysts and its capacity for germ line transmission. Embryonic stem cells may then be isolated from the transgenic animal.

The present invention also provides a genetically modified cell containing loss-of-function modification in at least one WFS1 allele as well as a genetically modified cell containing an expression cassette including a reporter gene functionally linked to a WFS1 expression regulatory nucleic acid sequence, and where the WFS1 expression regulatory nucleic acid sequence regulates an expression of the reporter gene. In one embodiment, such genetically modified cell may be obtained from a non-human animal produced in accordance with the methods of the present invention. In another embodiment, a genetically modified cell may be obtained by direct manipulation of primary or cultured cell (e.g., a central nervous system neuron, a peripheral nervous system neuron, a stem cell and a pancreatic β cell). Techniques for manipulating cells to create loss-of-function mutations are well known in the art.

In addition, the present invention provides a variety of isolated nucleic acids which may be useful in applications, such as producing a non-human animal and/or a genetically modified cell, compound screening assays, as well as treating and/or preventing motor neuron associated diseases.

“Nucleic acid” or “polynucleotide,” as used herein, refers to a nucleic acid, oligonucleotide, nucleotide, polynucleotide or any fragment thereof. It may be double-stranded or single-stranded DNA or RNA or DNA-RNA hybrid of genetic or synthetic origin, where the nucleic acid contains any combination of deoxyribo- and ribo-nucleotides and any combination of bases including, but not limited to, adenine, thymine, cytosine, guanine, uracil, inosine, xanthine hypoxanthine, etc., and combined with carbohydrate, lipid, protein or other materials. As used herein, “Complementary” nucleic acid refers to a nucleic acid molecule which is completely complementary to another nucleic acid or which will hybridize to such nucleic acid under conditions of high stringency. High stringency conditions are known in the art (see, for example, Maniatis, et al., Molecular Cloning: A Laboratory Manual (2d Ed.), 1989 and Ausubel, et al., Current Protocols in Molecular Biology, 2001, both of which are hereby incorporated by reference herein in their entirety). Stringent conditions are sequence-dependent and may be different in different circumstances. “cDNA,” as used herein, refers to an isolated polynucleotide, nucleic acid molecule or any fragment or derivative or complement thereof. It may be double-stranded or single-stranded, have originated recombinantly or synthetically, represent coding and/or noncoding 5′ and 3′ sequences. “Derivative” as used herein means a polynucleotide that has been subjected to a chemical modification or where a nontraditional base such as inosine. Derivative molecules may retain the biological characteristics of the naturally occurring molecules but may also possess additional features such as higher stability in vivo or enhanced activity. A polynucleotide “fragment” refers to a chain of consecutive nucleotides at least 12 or 19 nucleotides in length.

In one embodiment, the present invention provides a nucleic acid having essentially a nucleic acid sequence of SEQ ID NOS: 1, 2, 3, 4 and 5 or the complement thereof, or a functional fragment thereof, which includes at least 19 nucleotides. For example, a nucleic acid having essentially a nucleic acid sequence of SEQ ID NOS: 1, 2, 3, 4 and 5 may be used as an expression regulatory sequence to drive an expression of a transgene in a cell. If a patient/subject has a motor neuron associated disorder due to a loss-of-function WFS1 mutation in its genome, a copy of a functional WFS1 gene may be operably linked to a nucleic acid sequence of SEQ ID NOS: 1, 2, 3, 4 and 5 and introduced into the subject to treat motor neuron associated disorder. Because the expression of the WFS1 transgene is controlled by its “endogenous,” natural expression regulatory mechanism, normal (i.e., wild-type) expression pattern of WFS1 transgene is expected, which may help to reduce or minimize any potential adverse effects associated with the introduction of an foreign gene into a subject.

The isolated nucleic acid of the present invention may be an antisense nucleic acid or an interfering nucleic acid including, without limitation, an antisense RNA, an interfering RNA, a nucleic acid encoding an antisense RNA, a nucleic acid encoding an interfering RNA and vectors containing the same. Both antisense nucleic acid and interference nucleic acid may be produced in vitro, in vivo, ex vivo, or in situ, by chemical synthesis and/or through genetic engineering (e.g., using an expression vector).

Antisense RNA is an RNA molecule with a sequence complementary to a specific RNA transcript or mRNA, whose binding may prevent further processing of the transcript or translation of the mRNA. Antisense molecules may be generated, synthetically or recombinantly, with a nucleic-acid vector expressing an antisense gene-silencing cassette. Such antisense molecules may be single-stranded RNAs or DNAs, with lengths as short as 15-20 bases or as long as a sequence complementary to the entire mRNA. RNA molecules are generally sensitive to nucleases. To afford protection against nuclease digestion, an antisense deoxyoligonucleotide may be synthesized as a phosphorothioate, in which one of the nonbridging oxygens surrounding the phosphate group of the deoxynucleotide is replaced with a sulfur atom (Stein, et al., Oligodeoxynucleotides as inhibitors of gene expression: a review. Cancer Res., 48:2659-68, 1998).

Antisense molecules designed to bind to the entire mRNA or the entire 5′ UTR region may be made by inserting a cDNA or the 5′ UTP portion of a cDNA into an expression plasmid in the opposite or antisense orientation. Antisense molecules may also function by preventing translation initiation factors from binding near the 5′ cap site of the mRNA, or by interfering with interaction of the mRNA and ribosomes (e.g., U.S. Pat. No. 6,448,080, Antisense modulation of WRN expression; U.S. Patent Application No. 2003/0018993, Methods of gene silencing using inverted repeat sequences; U.S. Patent Application No., 2003/0017549, Methods and compositions for expressing polynucleotides specifically in smooth muscle cells in vivo; Tavian, et al., Stable expression of antisense urokinase mRNA inhibits the proliferation and invasion of human hepatocellular carcinoma cells. Cancer Gene Ther., 10:112-20, 2003; Maxwell and Rivera, Proline oxidase induces apoptosis in tumor cells and its expression is absent or reduced in renal carcinoma. J. Biol. Chem., e-publication ahead of print, 2003; Ghosh, et al., Role of superoxide dismutase in survival of Leishmania within the macrophage. Biochem. J, 369:447-52, 2003; and Zhang, et al., An anti-sense construct of full-length ATM cDNA imposes a radiosensitive phenotype on normal cells. Oncogene, 17:811-8, 1998).

Oligonucleotides antisense to a member of WFS1 signal transduction pathway (including WFS1) may be designed based on the nucleotide sequence of the member of interest. For example, a partial sequence of the nucleotide sequence of interest (generally, 15-20 base pairs) or a variation sequence thereof may be selected for the design of an antisense oligonucleotide. This portion of the nucleotide sequence may be within the 5′ UTR domain. A nucleotide sequence complementary to the selected partial sequence of the gene of interest or the selected variation sequence then may be chemically synthesized using one of a variety of techniques known to those skilled in the art including, without limitation, automated synthesis of oligonucleotides having sequences which correspond to a partial sequence of the nucleotide sequence of interest or a variation sequence thereof using commercially available oligonucleotide synthesizers, such as the Applied Biosystems Model 392 DNA/RNA synthesizer.

Once the desired antisense oligonucleotide has been prepared, its ability to modulate (e.g., reduce or enhance) WFS1 function and/or treat or prevent a motor neuron associated disorder then may be assayed. For example, the antisense oligonucleotide may be administered to a subject, such as a mouse or a human, and its effects on the disease may be determined using standard clinical and/or molecular biology techniques, such as Western-blot analysis and immunostaining.

It is within the confines of the present invention that oligonucleotides antisense to a member of WFS1 signal transduction pathway/system may be linked to another agent, such as a anti-apoptosis agent or ion-channel modulator. Moreover, antisense oligonucleotides may be prepared using modified bases (e.g., a phosphorothioate), as discussed above, to make the oligonucleotides more stable and better able to withstand degradation.

The nucleic acid of the present invention also may be an interfering RNA or RNAi, including small interfering RNA (siRNA). As used herein, “RNAi” refers to a double-stranded RNA (dsRNA) duplex of any length, with or without single-strand overhangs, wherein at least one strand, putatively the antisense strand, is homologous to the target mRNA to be degraded. As further used herein, a “double-stranded RNA” molecule includes any RNA molecule, fragment or segment containing two strands forming an RNA duplex, notwithstanding the presence of single-stranded overhangs of unpaired nucleotides. Additionally, as used herein, a double-stranded RNA molecule includes single-stranded RNA molecules forming functional stem-loop structures, such that they thereby form the structural equivalent of an RNA duplex with single-strand overhangs. The double-stranded RNA molecule of the present invention may be very large, comprising thousands of nucleotides; preferably, however, it is small, in the range of 21-25 nucleotides. In a preferred embodiment, the RNAi of the present invention comprises a double-stranded RNA duplex of at least 19 nucleotides.

In one embodiment of the present invention, RNAi may be produced in vivo by an expression vector containing a gene-silencing cassette coding for RNAi (see, e.g., U.S. Pat. No. 6,278,039, C. elegans deletion mutants; U.S. Patent Application No. 2002/0006664, Arrayed transfection method and uses related thereto; WO 99/32619, Genetic inhibition by double-stranded RNA; WO 01/29058, RNA interference pathway genes as tools for targeted genetic interference; WO 01/68836, Methods and compositions for RNA interference; and WO 01/96584, Materials and methods for the control of nematodes). In another embodiment of the present invention, RNAi is produced in vitro, synthetically or recombinantly, and transferred into a cell or a subject using standard molecular biology techniques. Methods of making and transferring RNAi are well known in the art (see, e.g., Ashrafi, et al., Genome-wide RNAi analysis of Caenorhabditis elegans fat regulatory genes. Nature, 421:268-72, 2003; Cottrell, et al., Silence of the strands: RNA interference in eukaryotic pathogens. Trends Microbiol., 11:37-43, 2003; Nikolaev, et al., Parc. A Cytoplasmic Anchor for p53. Cell, 112:29-40, 2003; Wilda, et al., Killing of leukemic cells with a BCR/ABL fusion gene RNA interference (RNAi). Oncogene, 21:5716-24, 2002; Escobar et al., RNAi-mediated oncogene silencing confers resistance to crown gall tumorigenesis. Proc. Natl. Acad. Sci. USA, 98:13437-42, 2001; and Billy et al., Specific interference with gene expression induced by long, double-stranded RNA in mouse embryonal teratocarcinoma cell lines. Proc. Natl. Acad. Sci. USA, 98:14428-33, 2001).

The present invention further provides methods and pharmaceutical compositions for treating or preventing a motor neuron associated disorder in a subject, including administering to the subject a pharmaceutical composition in an amount effective to enhance WFS1 function in the subject.

The inventor has shown that a motor neuron associated disease, such as ALS, may be caused by a WFS1 loss-of-function.

In one embodiment, a pharmaceutical composition of the present invention may include a pharmaceutically acceptable carrier and a nucleic acid which may function in vivo to correct or ameliorate a WFS1-dependent motor neuron disease including, without limitation, partially or completely correcting the underlining cause of the pathological conditions or ameliorating a symptom associated with the disorder. Examples of such nucleic acid include, but are not limited to, an expression vector carrying the nucleic acid of SEQ ID NOS: 1, 2, 3, 4 and 5 operably linked to a functional WFS1 transgene, an expression vector carrying a nucleic acid encoding a polypedite, such as an antibody (e.g., a single chain antibody and a scfv) which may enhance or inhibit WFS1-mediated signal transduction and an antisense nucleic acid and/or an interfering nucleic acid which may enhance or inhibit WFS1-mediated signal transduction.

Unless otherwise indicated, “polypeptide” shall include a protein, protein domain, polypeptide or peptide and any fragment or variant or derivative thereof having polypeptide function. The variants preferably have greater than about 75% homology with the naturally-occurring polypeptide sequence, more preferably have greater than about 80% homology, even more preferably have greater than about 85% homology and, most preferably, have greater than about 90% homology with the polypeptide sequence. In some embodiments, the homology may be as high as about 95%, 98%, or 99%. These variants may be substitutional, insertional or deletional variants. The variants may also be chemically modified derivatives: polypeptides which have been subjected to chemical modification, but which retain the biological characteristics of the naturally occurring polypeptide. In one embodiment of the present invention, the polypeptide is mutated such that it has a longer half-life in vivo.

As used herein, two polypeptide or nucleic acid sequences are said to be “identical” if the sequence of amino acid residues or nucleotides, respectively, in the two sequences is the same when aligned for optimum correspondence. Optimal alignment of sequences for comparison may be conducted by inspection or by using one of a number of algorithms, such as the homology alignment algorithm of Needleman and Wunsch (Needleman and Wunsch, A general method applicable to the search for similarities in the amino acid sequence of two proteins. J. Mol. Biol. 48: 443-53, 1970), the local homology algorithm of Smith and Waterman (Smith and Waterman, Comparison in biosequences. Adv. Appl. Math. 2: 482-9, 1981), the Pearson and Lipman algorithm (Pearson and Lipman, Improved tools for biological sequence comparison. Proc. Natl. Acad. Sci. USA 85: 2444-8, 1988), or computerized implementations of these algorithms (e.g., BLAST as offered by the National Center for Biotechnology Information). The degree of homology between two sequences is determined by comparing two optimally-aligned sequences, wherein the percentage is calculated by ascertaining the number of positions at which the identical amino acid residue or nucleic acid base occurs in both sequences, to yield the number of matched positions, dividing the number of matched positions by the total number of positions in the sequence, and multiplying the result by 100 to yield the percentage of sequence identity.

The WFS1 function in a cell or a subject may be enhanced or reduced at various levels, such as, the transcription level, the translation level, or the post-translation level. For example, if both alleles of WFS1 in a cell contain loss-of-function mutations, a pharmaceutical composition containing a expression vector, which may include a functional WFS1 gene operably linked to a WFS1 expression regulatory sequence, may be used to provide the cell with at least one copy of functional WFS1 gene and its protein products. In another example, if a cell contains at least one copy of functional WFS1 but its expression is suppressed, a pharmaceutical composition may be administered to enhance the expression of the endogenous WFS1 gene and/or reduce the suppression, e.g., by reducing the function of an endogenous inhibitor of WFS1 using an interfering RNA. The endogenous inhibitor of WFS1 may not necessarily be known in the art so long as an interfering RNA may be identified (e.g., by screening randomized interfering RNA library) which reduces the suppression of WFS1 function in a cell of interest. In addition, a pharmaceutical composition may contain a functional WFS1 gene operably linked to a strong promoter to enhance its expression and/or evade the suppression mechanism when introduced into a cell of interest. In one embodiment, a pharmaceutical composition of the present invention may contain a small molecule compound and/or a peptide/polypeptide (e.g., an antibody or a peptide mimic agent) or mimetic which may enhance WFS1 function or mimic WFS1 function and thus enhance WFS1-mediated signal transduction in a cell (e.g., a motor neuron).

Various protocols known in the art may be used to enhance WFS1 function in a subject. For example, WFS1 function may be directly enhanced in vivo, such as by administering to the subject a pharmaceutical composition in an amount effective to augment WFS1-mediated signal transduction. WFS1 function may also be enhanced using an ex vivo protocol. Under this approach, cells (particularly, stem cells) may be obtained from a subject in need of treatment. The stem cells may be genetically manipulated to include, for example, a functional WFS1 gene and optionally amplified in vitro. After being reintroduced back into the subject, these stem cells may proliferate and differentiate into motor neurons and therefore help to treat WFS1-mediated motor neuron associated diseases.

The pharmaceutically acceptable carrier may be “acceptable” in the sense of being compatible with the other ingredients of the composition and not deleterious to the recipient thereof. The pharmaceutically-acceptable carrier employed herein may be selected from various organic or inorganic materials that are used in pharmaceutical formulations and which may be incorporated as analgesic agents, buffers, binders, disintegrants, diluents, emulsifiers, excipients, extenders, glidants, solubilizers, stabilizers, suspending agents, tonicity agents, vehicles and/or viscosity-increasing agents. If necessary, pharmaceutical additives, such as antioxidants, aromatics, colorants, flavor-improving agents, preservatives and sweeteners, may also be added. Examples of acceptable pharmaceutical carriers include carboxymethyl cellulose, crystalline cellulose, nano-crystal, glycerin, gum arabic, lactose, magnesium stearate, methyl cellulose, powders, saline, sodium alginate, sucrose, starch, talc and water, among others.

The pharmaceutical composition of the present invention may be prepared by methods well known in the pharmaceutical arts. For example, the composition may be brought into association with a carrier or diluent, as a suspension or solution. Optionally, one or more accessory ingredients (e.g., buffers, flavoring agents, surface active agents and the like) also may be added. The choice of carrier will depend upon the route of administration of the composition. Formulations of the composition may be conveniently presented in unit dosage, or in such dosage forms as aerosols, capsules, elixirs, emulsions, eye drops, injections, liquid drugs, pills, powders, granules, suppositories, suspensions, syrup, tablets or troches, which can be administered orally, topically or by injection including, without limitation, intravenous, intraperitoneal, subcutaneous, intramuscular and intratumoral (i.e., direct injection into the tumor) injection.

The pharmaceutical composition is provided in an amount effective to treat the disorder in a subject to whom the composition is administered. As used herein, the phrase “effective to treat the disorder” means effective to ameliorate or minimize the clinical impairment or symptoms resulting from motor neuron associated disorder. For example, the clinical impairment or symptoms of ALS may be ameliorated or minimized by reducing/diminishing any pain or discomfort suffered by the subject; by extending the survival of the subject beyond that which would otherwise be expected in the absence of such treatment; or by inhibiting or preventing the development of the disorder.

The amount of pharmaceutical composition that is effective to treat a motor neuron associated disorder in a subject will vary depending on the particular factors of each case including, for example, the type or stage of the motor neuron associated disorder, the subject's weight, the severity of the subject's condition and the method of administration. These amounts can be readily determined by a skilled artisan.

In the method of the present invention, the pharmaceutical composition may be administered to a human or animal subject by known procedures including, without limitation, oral administration, parenteral administration (e.g., epifascial, intracapsular, intracutaneous, intradermal, intramuscular, intraorbital, intraperitoneal, intraspinal, intrasternal, intravascular, intravenous, parenchymatous or subcutaneous administration), transdermal administration and administration by osmotic pump. One preferred method of administration is parenteral administration, by intravenous or subcutaneous injection.

For oral administration, the formulation of the pharmaceutical composition may be presented as capsules, tablets, powders, and granules or as a suspension. The formulation may have conventional additives, such as lactose, mannitol, cornstarch or potato starch. The formulation also may be presented with binders, such as crystalline cellulose, cellulose derivatives, acacia, cornstarch or gelatins. Additionally, the formulation may be presented with disintegrators, such as cornstarch, potato starch or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the pharmaceutical composition may be combined with a sterile aqueous solution, which is preferably isotonic with the blood of the subject. Such a formulation may be prepared by dissolving a solid active ingredient in water containing physiologically-compatible substances, such as sodium chloride, glycine and the like, and having a buffered pH compatible with physiological conditions, so as to produce an aqueous solution, then rendering said solution sterile. The formulation may be presented in unit or multi-dose containers, such as sealed ampules or vials. The formulation also may be delivered by any mode of injection, including any of those described above. Where an infection or a neoplasm is localized to a particular portion of the body of the subject, it may be desirable to introduce the pharmaceutical composition directly to that area by injection or by some other means (e.g., by intra-tumoral delivery, by local delivery or by introducing the therapeutic composition into the blood or another body fluid).

For transdermal administration, the pharmaceutical composition may be combined with skin-penetration enhancers, such as propylene glycol, polyethylene glycol, isopropanol, ethanol, oleic acid, N-methylpyrrolidone and the like, which increase the permeability of the skin to the therapeutic composition and permit the pharmaceutical composition to penetrate through the skin and into the bloodstream. The pharmaceutical composition also may be further combined with a polymeric substance, such as ethyl cellulose, hydroxypropyl cellulose, ethylene/vinyl acetate, polyvinyl pyrrolidone and the like, to provide the composition in gel form, which may be dissolved in solvent, such as methylene chloride, evaporated to the desired viscosity and then applied to backing material to provide a patch. The pharmaceutical composition may be administered transdermally, at or near the site on the subject where the neoplasm is localized. Alternatively, the pharmaceutical composition may be administered transdermally at a site other than the affected area, in order to achieve systemic administration.

The pharmaceutical composition of the present invention also may be released or delivered from an osmotic mini-pump or other time-release device. The release rate from an elementary osmotic mini-pump may be modulated with a micro porous, fast-response gel disposed in the release orifice. An osmotic mini-pump would be useful for controlling release or targeting delivery, of the pharmaceutical composition.

In accordance with the method of the present invention, the pharmaceutical composition may be administered to a subject who has a motor neuron associated disease or disorder, either alone or in combination with one or more other drugs, such as anti-depression drugs, anti-apoptosis drugs and ion channel modulators. Additionally, when administered to a subject, the pharmaceutical composition may be combined with other anti-motor neuron associated disease therapies including, without limitation, surgical therapies and gene therapies.

The present invention further provides systems and methods for identifying a modulator of WFS1 in a motor neuron, including the steps of: (a) contacting a motor neuron with a candidate agent; and (b) determining an effect of the candidate agent on WFS1 function, where a change of the WFS1 function may indicate that the candidate agent is a modulator of WFS1 in a motor neuron. Unless otherwise indicated, the term “WFS1 function” refers to WFS1 biological activity and/or expression, as well as the activity and/or expression of an up-stream regulator of WFS1 or a down-stream effector of WFS1 in a WFS1 signal transduction pathway.

A candidate modulator agent may be any suitable factor or agent, which is capable of modulating WFS1 function including, without limitation, a small molecule compound, a peptide, a polypeptide, an antibody, a lipid, a saccharide, a polysaccharide, a nucleic acid (e.g., an expression vector, an antisense RNA or an interfering RNA) and combinations thereof. In one embodiment, the candidate modulator agent may be a small molecule organic compound. The system and method of the present invention is particularly suitable for screening a large library of candidate modulators. For example, a large number of candidate modulators (e.g., small molecule organic compounds) may be screened effectively and efficiently using systems of the present invention and high throughput screening techniques, where the change of WFS1 function may be identified by a high throughput screening detector, e.g., the identification of gene expression pattern using DNA array technology, or the identification of WFS1-mediated Ca²⁺ responses using a fluorescence detector.

The effect of a candidate agent on WFS1 function may be measured by determining its effect on a WFS1-mediated event. For example, it is known in the art that disruption of WFS1 gene may cause a cell undergoing apoptosis. Therefore, using a WFS1-mediated cell apoptosis model, the effect of a candidate modulator on WFS1 function may be determined, where at least a partial inhibition of the WFS1-mediated apoptosis may indicate that the candidate mimetic agent may be a modulator of WFS1. It has also been shown that WFS1 mediates calcium responses in a cell. Thus, the effect of a candidate modulator on WFS1 function may be measured by determining its effect on WFS1-mediated calcium responses in a cell (such as by labeling the cell with a calcium sensitive fluorescent indicator and detecting any change in fluorescent intensity), where at least a partial restoration of the WFS1-mediated calcium responses may indicate that the candidate modulator may be a modulator of WFS1. In addition, the inventor has shown that a non-human animal with a loss-of-function mutation in all WFS1 alleles develops a number of phenotypes/symptoms, which are similar to those of a motor neuron associated disease such as, for example, muscle wasting, denervation of the diaphragm, respiratory arrhythmias and psychiatric disorders. Accordingly, at least a partial inhibition or restoration of the WFS1-mediated phenotype may indicate the candidate mimic agent may be a WFS1 mimetic.

A candidate modulator of WFS1 function may be assayed using an in vitro, in vivo or ex vivo protocol. For example, an isolated motor neuron may be cultured in vitro, exposed to a candidate modulator and any effect of the candidate modulator on WFS1 may then be determined. In another example, a motor neuron may be cultured in vitro as part of a spinal cord slice, which provides an environment similar or closer to the in vivo environment. Alternatively, a candidate modulator may be administered to a subject. After a period sufficient for the contact of the candidate modulator with a motor neuron in vivo, the effect of WFS1 function in the motor neuron may then be determined either in situ (e.g., using an in situ imaging equipment (such as MRI) or an in situ histochemical technique) or in vitro by collecting and examining the treated motor neurons from the subject.

In one aspect, the present invention provides a method for identifying a WFS1 mimetic, including the steps of: (a) contacting a genetically modified cell with a candidate mimetic agent, wherein the genetically modified cell comprises an exon 8 related loss-of-function modification in all WFS1 alleles; and (b) determining an effect of the candidate mimic agent on WFS1-mediated signal transduction, wherein at least a partial restoration of the WFS1-mediated signal transduction function may indicate that the candidate agent may be a WFS1 mimetic.

A candidate mimetic agent may be any suitable factor or agent which is capable of mimicking WFS1 function including, without limitation, a small molecule compound, a peptide, a polypeptide, an antibody, a lipid, a saccharide, a polysaccharide, a nucleic acid and combinations thereof. In one embodiment, the candidate mimetic agent may be a small molecule organic compound. The effect of a candidate mimetic agent of WFS1 may be measured by determining its effect on a WFS1-mediated event, such as WFS1-mediated cell apoptosis, WFS1-mediated calcium responses in a cell, as well as WFS1-mediated phenotype in a subject. A candidate mimetic agent of WFS1 may be identified using an in vitro, in vivo or ex vivo protocol.

The present invention also provides a method for identifying a modulator of WFS1 expression, including the steps of: (a) contacting a cell with a candidate modulator agent, where the cell contains an expression cassette including a reporter gene functionally linked to a WFS1 expression regulatory nucleic acid sequence, and where the WFS1 expression regulatory nucleic acid sequence regulates an expression of the reporter gene; and (b) determining an effect of the candidate modulator agent on the reporter gene expression, where a change of the reporter gene expression indicates that the candidate modulator is a modulator of WFS1 expression.

A candidate modulator agent may be any suitable factor or agent which is capable of modulating WFS1 expression including, without limitation, a small molecule compound, a peptide, a polypeptide, an antibody, a lipid, a saccharide, a polysaccharide, a nucleic acid and combinations thereof. In one embodiment, the candidate modulator agent may be a small molecule organic compound.

A candidate modulator of WFS1 function may be identified using an in vitro, in vivo or ex vivo protocol. For example, a genetically modified cell (e.g., a motor neuron) which contains a WFS1 promoter-reporter gene construct may be cultured in vitro, exposed to a candidate modulator and any effect of the candidate modulator on the expression of the reporter gene may then be determined by examining the expression of the reporter gene. In another example, a genetically modified motor neuron which contains a WFS1 promoter-reporter gene construct may be cultured in vitro as part of spinal cord slice, which provides an environment similar or closer to the in vivo environment. Alternatively, a candidate modulator may be administered to a non-human animal of the present invention, which contains a plurality of genetically modified cells (e.g., a motor neuron), which includes a WFS1 promoter-reporter gene construct. After a period sufficient for the contact of the candidate modulator with the genetically modified cell, the effect of the candidate modulator on the expression of the reporter gene may then be determined either in situ (e.g., using an in situ imaging equipment (such as MRI) or an in situ histochemical technique) or in vitro by collecting and analyzing the genetically modified cells from the non-human animal.

Also provided is a method for validating an animal model for a motor neuron associated disorder, including the steps of (a) determining a plurality of phenotypes of a candidate genetically modified animal under a given condition; (b) determining a morphology and/or physiology of at least one motor neuron in the candidate genetically modified animal; (c) comparing the plurality of phenotypes with a plurality of phenotypes of an WFS1 model animal under the given condition, where the WFS1 model animal contains a plurality of genetically modified somatic cells having loss-of-function modification in at least one WFS1 allele; and (d) comparing the morphology and/or the physiology of the at least one motor neuron with a morphology of at least one motor neuron in the WFS1 model animal, where an substantially identical match between the plurality of phenotypes, the morphology and/or the physiology may indicate that the candidate generically modified animal may serve as a model animal for the motor neuron associated disorder. In one embodiment, the motor neuron associated disorder is ALS.

The present invention is described in the following Examples, which are set forth to aid in the understanding of the invention and should not be construed to limit in any way the scope of the invention as defined in the claims which follow thereafter.

### EXAMPLES

### Example 1

### Drug Target Finding and Validation Scheme and Identification of MWFS1 as a Regulated Gene Product in Brain

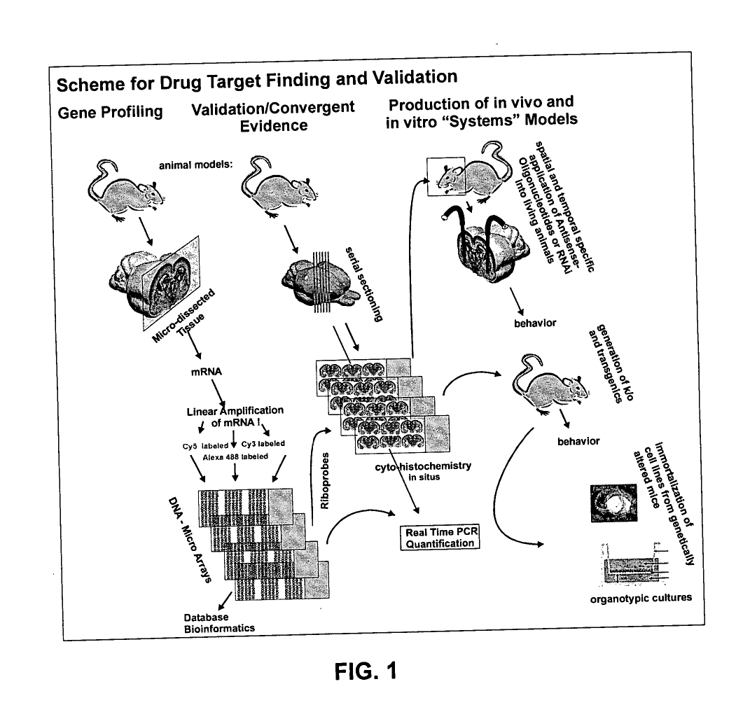

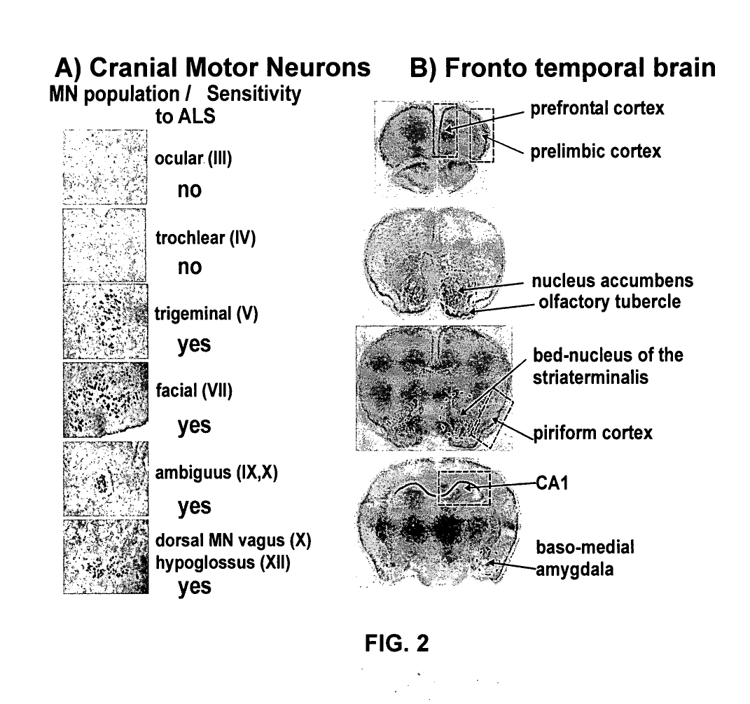

One goal of studying animal models of central nervous system associated disorder indications is to identify molecular substrates of disease associated, pathological behavior and ultimately to aid in the development of drugs to prevent or treat such disorders. One of the approaches used by researchers, expression profiling, is to examine the relative levels of mRNAs found in specific areas of the brain. This approach is based on the premise that specific mRNA abundances are altered because of disease state. Using this approach, the inventor has established a technology platform, which allows quantitative analysis of changes in the transcriptome in selected brain loci of experimental and control animals through expression profiling using cDNA micro arrays and to validate such changes in vitro and in vivo (FIG. 1).

In brief, specific brain loci of experimental and control animals are micro-dissected by Palkovits “punch” method or by laser capturing(s). RNA is prepared from the sample and amplified by linear amplification. Complex cDNA probes are generated by fluorescent labeling. Multiplexed probes, labeled with different fluorescent dyes, are hybridized to the same cDNA micro array simultaneously. Reproducible expression changes, which are detected by gene expression profiling, are confirmed by quantitative PCR and/or quantitative in situ hybridization. Data from human linkage studies and/or other animal models for the same or similar indication may be used as corroborating and convergent evidence for the significance of a candidate gene. Candidates that passed these stringent screenings are then manipulated in vivo to unravel changes in behavior, which, are correlated with the gain or loss-of-function of the gene under investigation.

Following this scheme, the inventor discovered that wfs1 mRNA is down regulated in the prefrontal cortex (PFC) of mice reared in isolation as compared to animals reared in social groups and in enriched environments, an animal model for anxiety and depression. He obtained convergent evidence for the correlation of changes in wfs1 expression with changes in behavior from an animal model for psychosis based on chronic phencyclidine injection. Here, wfs1 is up regulated specifically in the olfactory tubercle of experimental animals as compared to control animals.

Further investigation by the inventor, as detailed below, led to the discovery that wfs1 possesses crucial functionality for the maintenance and physiology of somatic motor neurons and could embody similar functions in select neuronal populations in the temporal frontal brain. Experimental corruption of this function leads in mice to a model of amyotrophic lateral sclerosis (ALS) and other motor neuron diseases.

### Example 2

### WFS1 Expression in the CNS

The inventor raised guinea pig antisera against the amino and carboxyl terminal portions of the protein and investigated the regional distributions of WFS1 in the fronto temporal brain, the hindbrain and the spinal cord by a combination of in situ hybridization and immuno-histochemical analysis with a single cell resolution. The inventor found prominent and regional specific expression in the fronto temporal brain, in particular in layer 2 of the prefrontal cortex, the prelimbic cortex, the piriform cortex as well as in the nucleus accumbens, the bed nucleus of the striaterminalis, the olfactory tubercle, the baso-medial amygdala and the CA1 region of the hippocampus (FIG. 2). This expression pattern indicates that WFS1 may function in cognitive, emotional and visceral control of behavior. Dysfunction of any or all of these areas could conceivably contribute to the manifestation of the cognitive decline observed in ALS patients with fronto-temporal dementia. In fact, neuronal atrophy in cortical layer 2 has been observed in ALS ftd patients and behavioral abnormalities, such as depression, memory loss, assaultive behavior, anxiety and/or panic attacks, hallucinations and suicide attempts, are clinical hallmarks of WS. Within the hindbrain, the inventor found that the expression of WFS1 was limited to motor neuron (“MN”). Most interestingly, the inventor found that all ALS sensitive cranial MNs express WFS1, where ocular and trochlear MNs, which appear to be resistant to ALS, do not express WFS1 (FIG. 2).

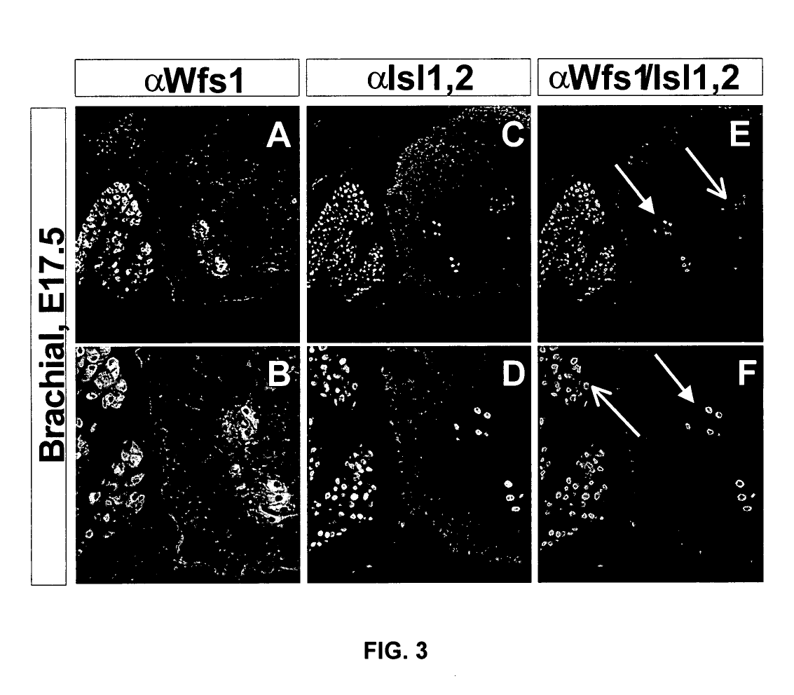

Within the spinal cord, WFS1 appeared to be expressed in virtually all somatic MNs (solid arrow, FIG. 3E) but not in preganglionic MNs (open arrow, FIG. 3E). WFS1 was also expressed in 1a afferent neurons of the dorsal root ganglia (FIG. 3A) as identified by costaining with anti-parvalbumin antiserum. The expression of WFS1 in somatic MNs began at around E17.5 and appeared to be maintained throughout adulthood.

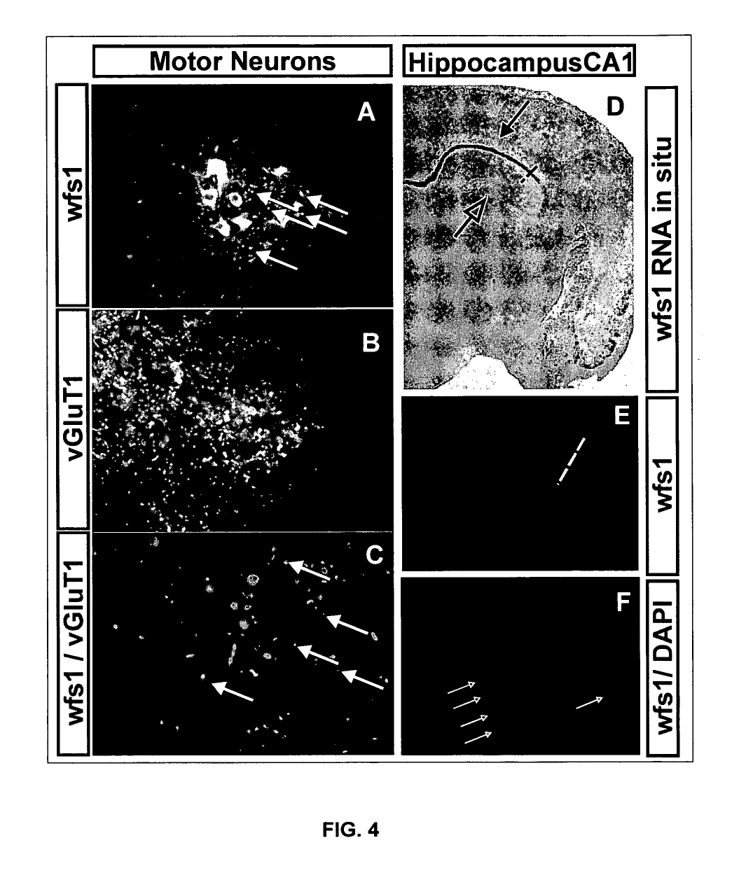

The inventor also investigated the subcellular localization of wolframin in MNs and CA1 hippocampal neurons. There was robust expression of wolframin in the soma of both types of neurons, which is consistent with previously described localization of wolframin (solid arrows, FIGS. 3F, 4E, and 4F). In addition, the inventor noticed a wolframin specific punctate staining within the area of MN pools in ventral horns of the spinal cord (solid arrows, FIG. 3A). Co-staining with anti vesicular glutamate transporter (vGluT1) antisera, a presynaptic marker for excitatory synapses, revealed that many wolframin puncta were juxtaposed in close proximity to vGluT1 puncta specifically within the area of MN pools (FIG. 4C, 600× magnification). These observations suggest an enrichment of wolframin in postsynaptic specializations in MN dendrites and are consistent with the inventor's findings that Wolframin is present in dendrites of CA1 neurons (FIG. 4D). The inventor shown that the WFS1 expression pattern exhibited a remarkable overlap with the anatomical substrate of ALS-FTD.

### Example 3

### The Production of Systemic WFS1 Loss-of-Function Mice and Mouse Lines that Overexpress WFS1 in Adult MN

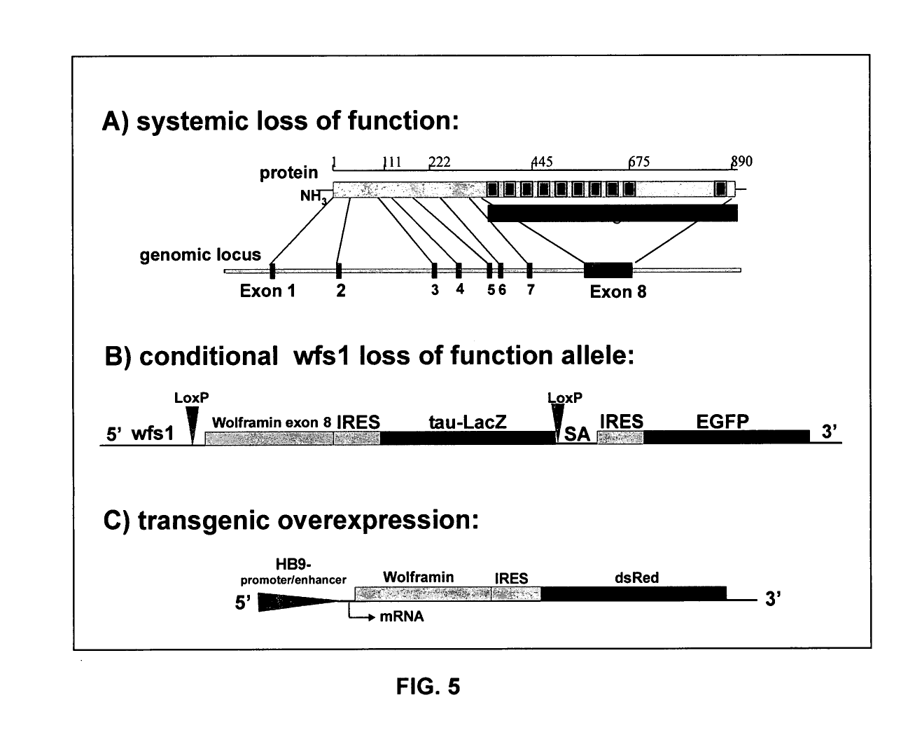

FIG. 5 shows the design of a systemic loss-of-function allele, a conditional loss-of-function allele, and a transgenic wfs1 over expression vector from which wfs1 is expressed together with the marker protein EGFP under the transcriptional control of the Hb9 promoter and enhancer sequences. All gene targeting work was carried out in C57B1/6 derived ES cells and recombinant ES cells were injected into B1/6 blastocysts. The strain origin of the ES cells (C57B1/6) and the particular strain origin of the blastocysts (C57B1/6) for ES cell injections were chosen purposefully for two reasons: a) to minimize the possibility for the development of a pancreatic phenotype since it has been shown previously that the mouse strain C57B1/6 is particularly resistant to pancreatic dysfunction; and b) to allow for robust behavioral testing of the resulting mutant animals since it is known that mixed strain backgrounds can impact negatively on the quality of behavioral analysis in mice. It has been previously shown that the homeobox gene Hb9 promoter and enhancer confer MN specificity to the transgenic expression of EGFP or dsRed.

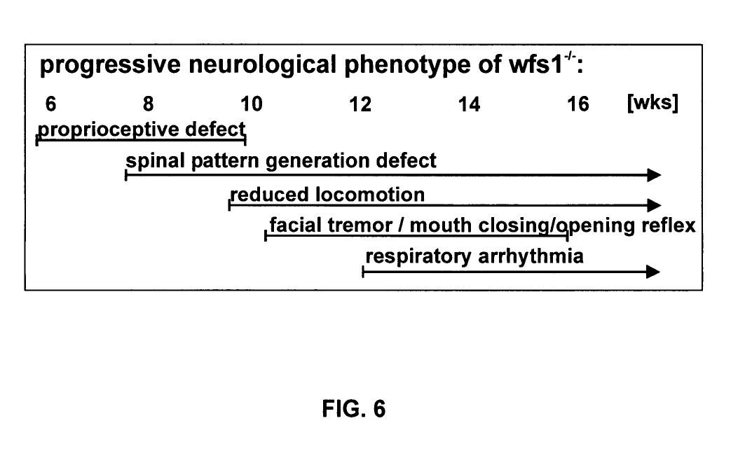

The systemic WFS1 loss-of-function animals were tested for the appearance of general neurological signs, for a motor phenotype as revealed by the accelerating rotarod paradigm, and for pancreatic dysfunction by measuring blood glucose levels in a longitudinal assay design with all measures taken once every week. The animals exhibit a progressive neurological motor phenotype beginning with 6 weeks of age (FIG. 6) in the absence of any signs of hyperglycemia. In particular, in wfs1 wt, and in heterozygous and homozygous wfs1 loss-of-function animals blood glucose levels remain at 110 mg/ml +/−24 mg/ml to their death at about 8 months of age. The inventor confirmed that the absence of a pancreatic dysfunction in wfs1 loss-of-function mice is a function of strain background by interbreeding the C57B1/6 wfs1 loss-of-function animals with mice of the 129 strain. In the offspring of these crosses wfs1 loss-of-function is correlated with the progressive elevation of blood glucose levels from an average of 100 mg/ml at 4 months of age to 340 mg/ml, a sign of overt hyperglycemia, at 6 months of age.

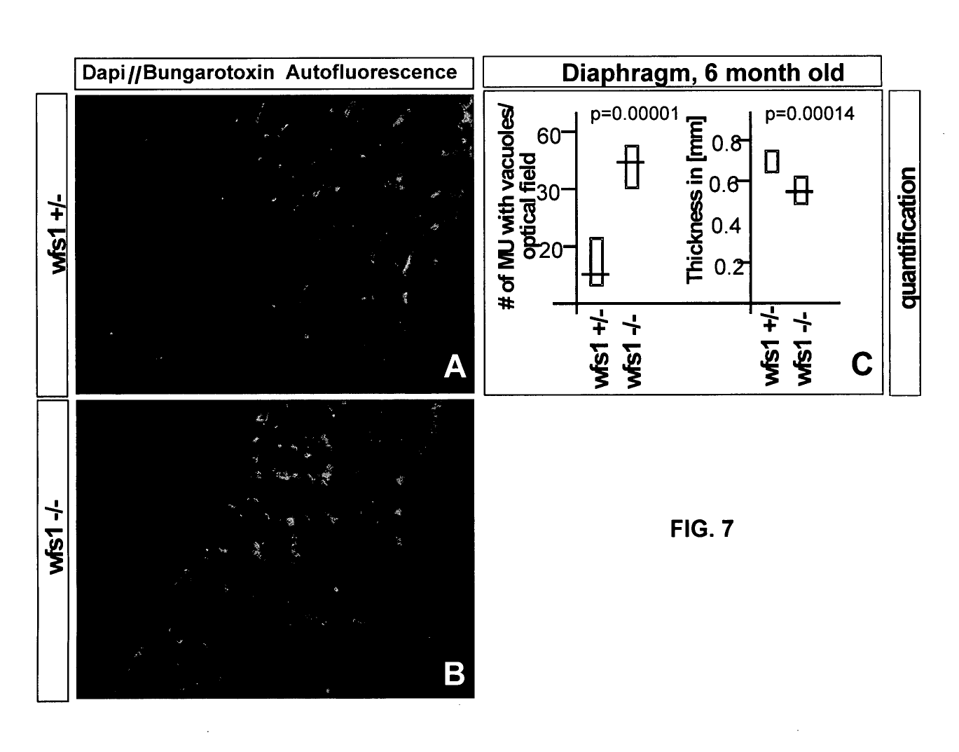

Between 6 and 8 weeks of age, the inventor observed a transient limb clutching phenotype. Often the observation of limb clutching is indicative of a spinal cord motor neuron or proprioceptive defect. At 8 weeks of age the inventor first noticed a pattern generation defect affecting the hindlimbs only: Whereas the wild-type animals display normal walking pattern formation and climb over obstructions in their way by lifting limbs in an alternating pattern, the WFS1−/− mice dragged both hindlimbs and navigated obstructions by pulling both hindlimbs over simultaneously. If challenged on the accelerating “rotarod” WFS1 −/− mice exhibited a progressive decline of motor abilities with an onset of phenotype at about 60 days of age. At about 100 days of age, WFS1 −/− animals performed rather poorly on the “rotarod” and repeated exposure to the test (1 hour intertest interval) did not improve their performance. However, the WFS1 +/−animals exhibit a marked ability to adapt to the test with repeated exposure. This observation could point to the possibility that both learning and memory systems as well as motor systems were affected at this time in the WFS1 −/− animals, whereas in the WFS1 +/−animals the deficit lies within the motor system only. Between 10 and 14 weeks, the inventor observed transient facial tremors and persistent, repetitive mouth closing and opening leading to an audible “clicking” sound made by the WFS1 −/− animals. By 12 weeks of age, WFS1 −/− animals developed abnormal breathing leading to arrhythmia and to very pronounced, intermittent gasping followed by up to 15 seconds of no inspiration activity by 6 months of age. Histochemical analysis of euthanized animals of 6 months of age revealed severely atrophied skeletal muscles in WFS1 loss-of-function animals. For example, the diaphragm muscle was thinned, nearly all motor units exhibited large vacuoles and many neuromuscular junctions (NMJ) were fragmented and appeared discontinuous (FIGS. 7A, B: Cross section through diaphragm, open arrow points to a disintegrating NMJ revealed by a-Bungarotoxin staining; C: quantification of numbers of vacuolized motor units and muscle thickness of WFS1 1+/− and wfs 1 −/− animals).

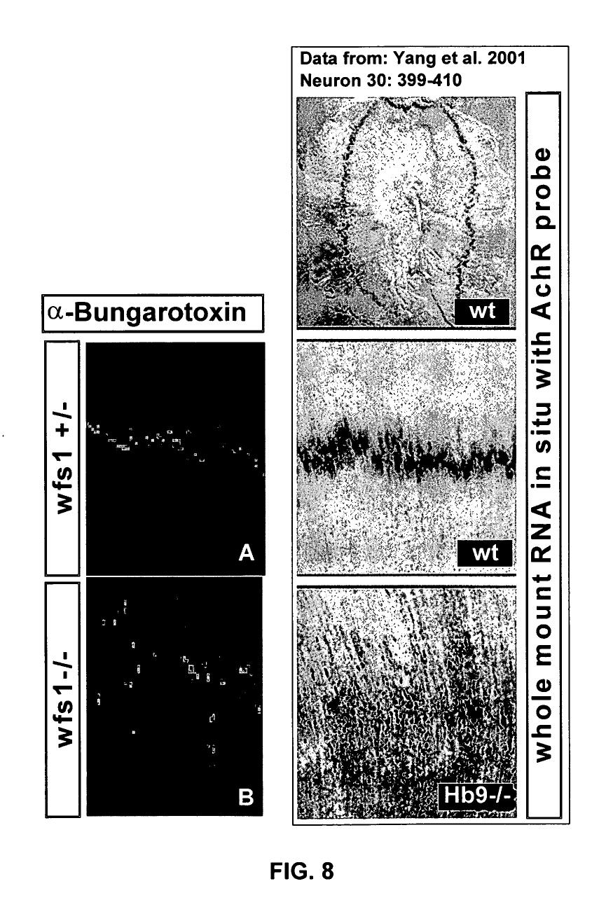

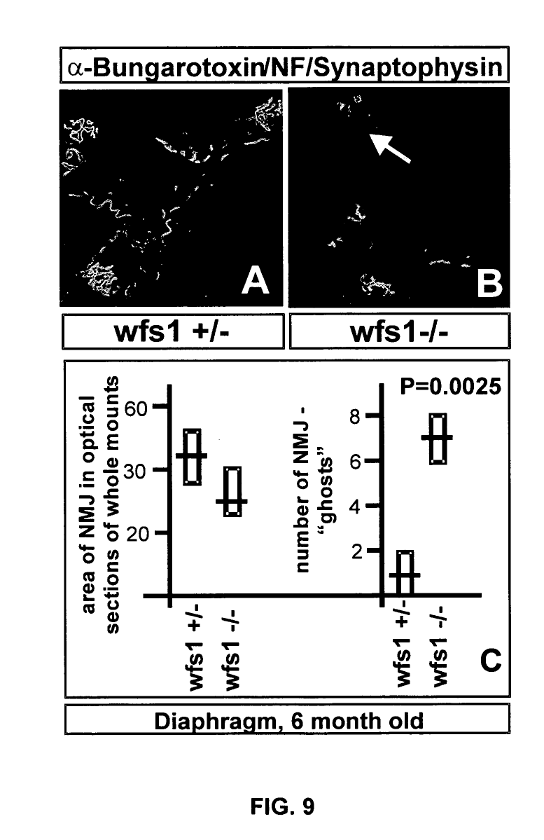

Absence of neuronal innervation of the diaphragm in Hb9−/− mice has been associated with a broadening of the band of acetylcholine receptor (AchR) clusters, which is located centrally at the equator of each motor unit. Interestingly, WFS1 loss-of-function lead to a dispersion of AchR clusters in the diaphragm resembling the pattern observed in Hb9−/− mice (FIG. 8: diaphragm analysis of whole mount preparations, A, B: staining of AchR clusters with Cy3-conjugated a-Bungarotoxin). These data are included to illustrate the similarities in the pattern of AchR clusters caused by the absence of MN axons in the Hb9−/− mice and in the wfs1−/− mice. Further analysis of the structural integrity of NMJs with presynaptic (neurofilament and synaptophysin) and postsynaptic (AchR) markers revealed direct evidence for a partial denervation of the diaphragm muscle and a reduction in size and complexity of NMJs in WFS1 −/− mice (FIGS. 9: A, B: NMJs in WFS1 +/− and in WFS1 −/− animals, respectively, C: quantification of relative area of NMJs and number of fully denervated, a-bungarotoxin positive AchR clusters, so called “NMJ ghosts”, per 10 optical fields).

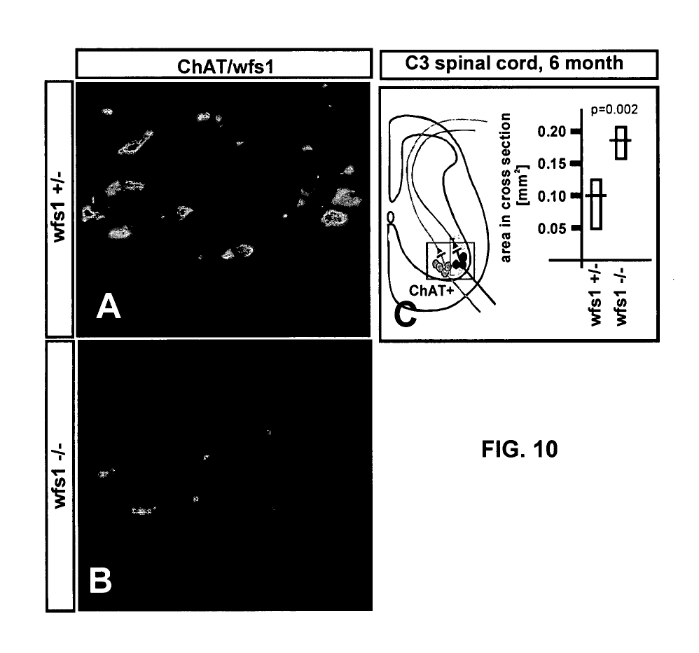

Within the spinal cord, the inventor observed a severe swelling of M soma (FIGS. 10: A, B: spinal MN a level C3 defined by their position in ventral horns and expression of ChAT, C: quantification of MN soma area on cross sections). Ultra structural analysis of swollen MN often found the presence of cytoplasmatic and mitochondrial micro-vacuoles. Mitochondrial swelling can lead to the rupture of the outer mitochondrial membrane and the subsequent release of cytochrome C. Therefore, neuronal swelling is believed to be a morphological marker of neuronal dysfunction and an early indication of apoptotic death of neurons.

On WFS1's effect on apoptosis, the inventor found that at 6 months of age there are 20% more spinal motor neurons in WFS1 −/− animals as compared to wfs1−/+animals. This result suggests that the lack of WFS1 may be anti-apoptotic and protects MNs from apoptosis. This interpretation is supported by the results derived from the ectopic expression of WFS1 in frog oocytes, which leads to an increase of the steady state levels of cytosolic Ca²⁺. Sustained, increased levels of cytosolic Ca²⁺ are considered pro-apoptotic. Conversely, a loss-of-function of WFS1 might lead to a decrease in the steady state levels of cytosolic Ca²⁺ and thus to an anti-apoptotic effect. In light of this result, the inventor suggested that although WFS1 loss-of-function mutations may protect MNs from natural cell death during MN ontogeny, it may lead the impairment in Ca²⁺ dependent signal transduction and therefore causing muscle wasting and loss of trophic support of MNs, which further exacerbates MN dysfunction.

As described above, blood glucose levels were normal (86 to 134 mg/ml) and do not differ as a function of WFS1 gene dosage up to death in the C57B1/6 strain. Recently, however, scientists have reported on the development of WFS1 loss-of-function animals (see, e.g., Ishihara, et al.), which exhibit progressive beta-cell loss and impaired stimulus-secretion coupling in insulin secretion. Hum Mol. Genet. 13:1159-70, 2004). These researchers observed the manifestation of overt diabetes at 32 weeks of age in their animals. However, there has been no report on the finding of a neuronal, or in particular, a motor neuronal phenotype in those knockout animals. It is noteworthy that both, the particular mouse strain utilized in these studies as well as the physical germline modification of the wfs1 locus is entirely different from the described invention. Ishihara et. al. produced their particular modification of the endogenous wfs1 gene in ES cell lines derived from the 129 mouse strain. They then analyzed the resulting phenotype in a mixed strain background resulting from 129×C57/B16 crosses. The germline modification these researchers produced deleted exon 2 only, a small exon coding 78 amino acids of the immediate N terminus of wolframin protein. Ishihara et al. observed the presence of nearly full length WFS1 mRNA in their knockout animals. It is known that there is an initiation codon at the beginning of exon 3, which is in frame with the rest of the coding sequence of WFS1 and could potentially lead to the expression of a truncated form of wolframin of 810 amino acids, compared to the 890 amino acids of wild type protein. Therefore, it appears possible that the genetic modification of the WFS1 locus produced by Ishihara, et al., resulted in a weaker phenotype with only pancreatic involvement but no neuronal or, in particular, motor neuronal component.

In summary, the inventor's study indicates that WFS1 dysfunction in neurons could play a role in ALS-FTD. The results further suggest that the systemic WFS1 −/− animals might be an informative model in which neurological and cell physiological aspects of ALS-FTD can be studied while not confounded by an underlying pancreatic dysfunction.

The inventor also produced a conditional loss-of-function allele for wfs1 that allows both, the tissue specific ablation of wfs1 as well as the immunohistochemical detection of cells that are either wt, het or mut for wfs1. FIG. 5B depicts the principle features of the conditional wfs1 loss-of-function allele. The ablation of gene function is achieved by cre mediated DNA site specific recombination. In the unrecombined case a biscistronic mRNA is transcribed which encodes a functional, full length wfs1 protein followed by a IRES tauLacZ marker cassette. In the cre recombined case, exon 8 and the tauLacZ marker is deleted and instead a mRNA is produced, which encodes the first 37% of the wfs1 protein followed by an IRES-EGFP marker cassettes. The translation of the truncated wfs1 gene leads to an unstable and dysfunctional protein product. In the unrecombined case, the marker protein expression of tauLacZ will stain the soma and axon of all cells that express wfs1, however, after cre mediated recombination, wfs1 expressing cells will express the marker protein EGFP. Hence the gene dosage of wfs1 can be assessed at a single cell resolution in histological preparations by monitoring expression of the two marker proteins by which a) the exclusive expression of the lacZ marker signifies functional wfs1 expression from both alleles, b) the exclusive expression of the marker protein EGFP signifies ablation of functional wfs1 expression from both alleles (complete loss-of-function) and c) coexpression of the marker proteins lacZ and EGFP signifies a cre mediated, wfs1 ablation on only one allele (heterozygous loss-of-function configuration).

All publications, references, patents and patent applications cited herein are incorporated by reference in their entirety to the same extent as if each individual application, patent or patent application was specifically and individually indicated to be incorporated by reference in its entirety.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed:

1. A non-human animal comprising a plurality of genetically modified cells, wherein the plurality of genetically modified cells comprises a loss-of-function modification in at least one WFS1 allele.

2. The non-human animal of claim 1, wherein the non-human animal is a rodent.

3. The non-human animal of claim 1, wherein the rodent is a mouse.

4. The non-human animal of claim 1, wherein at least one of the plurality of genetically modified cells is selected from the group consisting of a central nervous system neuron, a peripheral nervous system neuron, a stem cell, and a pancreatic β cell.

5. The non-human animal of claim 1, wherein at least one of the plurality of genetically modified cells is a motor neuron.

6. The non-human animal of claim 1, wherein the loss-of-function modification is a complete loss-of-function modification.

7. The non-human animal of claim 1, wherein the loss-of-function modification is an exon 8 related loss-of-function modification.

8. The non-human animal of claim 7, wherein the exon 8 related loss-of-function modification is a mutation of an exon 8 related nucleic acid sequence selected from the group consisting of a deletion mutation, a frame shift mutation, a nonsense mutation, a missense mutation, and a splice donor site mutation.

9. The non-human animal of claim 8, wherein the exon 8 related nucleic acid sequence comprises exon 8 of WFS1 gene.

10. The non-human animal of claim 1, wherein the plurality of genetically modified cells are homozygous WFS1 loss-of-function cells.

11. The non-human animal of claim 1, wherein the non-human animal displays no pancreatic related phenotype until at least about six months of age.

12. The non-human animal of claim 11, wherein the pancreatic related phenotype comprises a change in glucose level from about 100 mg/ml to about 340 mg/ml.

13. An offspring of the non-human animal of claim 1.

14. A genetically modified somatic cell obtained from the non-human animal of claim 1.

15. A non-human animal comprising a plurality of genetically modified somatic cells, wherein the plurality of genetically modified somatic cells comprises an expression cassette, wherein the expression cassette comprises a reporter gene functionally linked to a WFS1 expression regulatory nucleic acid sequence and wherein the WFS1 expression regulatory nucleic acid sequence regulates an expression of the reporter gene.

16. The non-human animal of claim 15, wherein the non-human animal is a rodent.

17. The non-human animal of claim 15, wherein the rodent is a mouse.

18. The non-human animal of claim 15, wherein at least one of the plurality of genetically modified somatic cells is selected from the group consisting of a central nervous system neuron, a peripheral nervous system neuron, a stem cell, and a pancreatic β cell.

19. The non-human animal of claim 15, wherein at least one of the plurality of genetically modified somatic cells is a motor neuron.

20. The non-human animal of claim 15, wherein the WFS1 expression regulatory nucleic acid sequence comprises a nucleic acid sequence of SEQ ID NOS: 1, 2, 3, 4 and 5.

21. The non-human animal of claim 15, wherein the reporter gene comprises a nucleic acid sequence encoding a fluorescent polypeptide.

22. The non-human animal of claim 15, wherein the plurality of genetically modified somatic cells further comprises an exogenous tissue-specific expression controlling element, wherein the exogenous tissue-specific expression controlling element regulates an expression of the reporter gene.

23. An offspring of the non-human animal of claim 15.

24. A genetically modified somatic cell obtained from the non-human animal of claim 15.

25. A genetically modified cell comprising a loss-of-function modification in at least one WFS1 allele.

26. The genetically modified cell of claim 25, wherein the cell is a rodent cell.

27. The genetically modified cell of claim 25, wherein the rodent cell is a mouse cell.

28. The genetically modified cell of claim 25, wherein the cell is selected from the group consisting of a central nervous system neuron, a peripheral nervous system neuron, a stem cell, and a pancreatic β cell.

29. The genetically modified cell of claim 25, wherein the cell is a motor neuron.

30. The genetically modified cell of claim 25, wherein the loss-of-function modification is a complete loss-of-function modification.

31. The genetically modified cell of claim 25, wherein the loss-of-function modification is an exon 8 related loss-of-function modification.

32. The genetically modified cell of claim 23, wherein the genetically modified cell is an embryonic stem cell.

33. The genetically modified cell of claim 25, wherein the exon 8 related loss-of-function modification is a mutation of an exon 8 related nucleic acid sequence selected from the group consisting of a deletion mutation, a frame shift mutation, a nonsense mutation, a missense mutation and a splice donor site mutation.

34. The genetically modified cell of claim 25, wherein the exon 8 related nucleic acid sequence comprises exon 8 of WFS1 gene.

35. The genetically modified cell of claim 25, where the cell is homozygous WFS1 loss-of-function cell.

36. A genetically modified cell comprising an expression cassette, wherein the expression cassette comprises a reporter gene functionally linked to a WFS1 expression regulatory nucleic acid sequence and wherein the WFS1 expression regulatory nucleic acid sequence regulates an expression of the reporter gene.

37. The genetically modified cell of claim 36, wherein the genetically modified cell is selected from the group consisting of a central nervous system neuron, a peripheral nervous system neuron, a stem cell and a pancreatic β cell.

38. The genetically modified cell of claim 36, wherein the genetically modified cell is a motor neuron.

39. The genetically modified cell of claim 36, wherein the genetically modified cell is an embryonic stem cell.

40. The genetically modified cell of claim 36, wherein the WFS1 expression regulatory nucleic acid sequence comprises a nucleic acid sequence of SEQ ID NOS: 1, 2, 3, 4 and 5.

41. The genetically modified cell of claim 36, wherein the reporter gene comprises a nucleic acid sequence encoding a fluorescent polypeptide.

42. The genetically modified cell of claim 36, wherein the plurality of genetically modified somatic cells further comprises an exogenous tissue-specific expression controlling element, wherein the exogenous tissue-specific expression controlling element regulates an expression of the reporter gene.

43. An expression vector comprising a reporter gene functionally linked to a WFS1 expression regulatory nucleic acid sequence, wherein the WFS1 expression regulatory nucleic acid sequence regulates an expression of the reporter gene.

44. The expression vector of claim 43, wherein the WFS1 expression regulatory nucleic acid sequence comprises a nucleic acid sequence of SEQ ID NOS: 1, 2, 3, 4 and 5.

45. The expression vector of claim 43, wherein the reporter gene comprises a nucleic acid sequence encoding a fluorescent polypeptide.

46. The expression vector of claim 43, wherein the expression vector further comprises an exogenous tissue-specific expression controlling element, wherein the exogenous tissue-specific expression controlling element regulates an expression of the reporter gene.

47. An isolated nucleic acid selected from the group consisting of a nucleic acid having essentially a nucleic acid sequence of SEQ ID NOS: 1, 2, 3, 4 and 5, a nucleic acid having essentially a nucleic acid sequence which is complementary of SEQ ID NOS: 1, 2, 3, 4 and 5, and a functional fragment thereof, which comprises at least 19 nucleotides.

48. The isolated nucleic acid of claim 47, wherein the isolated nucleic acid is selected from the group consisting of an antisense nucleic acid and an interfering nucleic acid.

49. The isolated nucleic acid of claim 48, wherein the isolated nucleic acid encodes a nucleic acid selected from the group consisting of an antisense RNA and an interfering RNA.

50. The isolated nucleic acid of claim 48, wherein the isolated nucleic acid comprises a plurality of nucleotides and wherein at least one of the plurality of nucleotides is modified.

51. A vector comprising a nucleic acid sequence encoding the isolated nucleic acid of claim 48.

52. A pharmaceutical composition comprising the isolated nucleic acid of claim 41 and a pharmaceutically acceptable carrier.

53. An isolated double-stranded nucleic acid consisting essentially of the isolated nucleic acid of claim 47 and the complementary nucleic acid thereof.

54. An isolated nucleic acid comprising an interfering nucleic acid for inhibiting an expression of WFS1 gene in a cell.

55. The isolated nucleic acid of claim 54, wherein the interfering nucleic acid is an interfering RNA.

56. The isolated nucleic acid of claim 54, wherein the interfering nucleic acid encodes an interfering RNA.

57. The isolated nucleic acid of claim 56, wherein the isolated nucleic acid is an expression vector.

58. The isolated nucleic acid of claim 54, wherein the cell is selected from the group consisting of a chicken cell, a rodent cell, and a human cell.

59. The isolated nucleic acid of claim 54, wherein the cell is selected from the group consisting of a central nervous system neuron, a peripheral nervous system neuron, a stem cell and a pancreatic β cell.

60. The isolated nucleic acid of claim 59, wherein the cell is a motor neuron.

61. The isolated nucleic acid of claim 60, wherein the motor neuron is in a spinal cord slice.

62. The isolated nucleic acid of claim 54, wherein the cell is in a subject.

63. The isolated nucleic acid of claim 62, wherein the subject is a rodent.

64. The isolated nucleic acid of claim 63, wherein the rodent is a mouse.

65. The isolated nucleic acid of claim 62, wherein the subject is a human.

66. A pharmaceutical composition comprising the isolated nucleic acid of claim 47 and a pharmaceutically acceptable carrier.

67. A method for treating or preventing a motor neuron associated disorder in a subject, comprising administering to the subject a pharmaceutical composition in an amount effective to enhance WFS1 function in the subject.

68. The method of claim 67, wherein the motor neuron associated disorder is a progressive motor neuron degeneration disorder.

69. The method of claim 67, wherein the motor neuron associated disorder is amyotrophic lateral sclerosis (ALS).

70. The method of claim 67, wherein the WFS1 function is enhanced using at least one protocol selected from the group consisting of a transcription level enhancement, a translation level enhancement and a post-translation level enhancement.

71. The method of claim 67, wherein the WFS1 function is enhanced using at least one protocol selected from the group consisting of an in vivo enhancement and an ex vivo enhancement.

72. The method of claim 67, wherein the subject is a rodent.

73. The method of claim 72, wherein the rodent is a mouse.

74. The method of claim 67, wherein the subject is a human.

75. The method of claim 70, the WFS1 function is enhanced in a cell in the subject and wherein the cell is selected from the group consisting of a central nervous system neuron, a peripheral nervous system neuron, a stem cell, and a pancreatic p cell.

76. The method of claim 67, wherein the cell is a motor neuron.

77. The method of claim 67, wherein the cell is an embryonic stem cell.

78. A method for identifying a modulator of WFS1 expression, comprising:
(a) contacting a cell with a candidate agent, wherein the cell comprises a reporter gene functionally linked to a WFS1 expression regulatory nucleic acid sequence; and
(b) measuring the expression of the reporter gene, wherein a change in expression of the reporter gene indicates that the candidate agent is a modulator of WFS1 expression.

79. The method of claim 78, wherein the cell is selected from the group consisting of a central nervous system neuron, a peripheral nervous system neuron, a stem cell and a pancreatic β cell.

80. The method of claim 78, wherein the cell is a motor neuron.

81. The method of claim 80, wherein the motor neuron is in a spinal cord slice.

82. The method of claim 78, wherein the cell is in a subject.

83. The method of claim 82, wherein the subject is a rodent.

84. The method of claim 78, wherein the WFS1 expression regulatory nucleic acid sequence comprises a nucleic acid sequence of SEQ ID NOS: 1, 2, 3, 4 and 5.

85. The method of claim 78, wherein the reporter gene comprises a nucleic acid sequence encoding a fluorescent polypeptide.

86. A method for identifying a WFS1 mimetic, comprising:
(a) contacting a genetically modified cell with a candidate agent, wherein the genetically modified cell comprises a loss-of-function modification in at least one WFS1 allele; and
(b) determining an effect of the candidate agent on WFS1-mediated signal transduction, wherein at least a partial restoration of the WFS1-mediated signal transduction indicates that the candidate agent is a WFS1 mimetic.

87. The method of claim 86, wherein the genetically modified cell is selected from the group consisting of a central nervous system neuron, a peripheral nervous system neuron, a stem cell, and a pancreatic β cell.

88. The method of claim 86, wherein the genetically modified cell is a motor neuron.

89. The method of claim 88, wherein the motor neuron is in a spinal cord slice.

90. The method of claim 86, wherein the genetically modified cell is in a subject.

91. The method of claim 90, wherein the subject is a rodent.

92. The method of claim 91, where the subject is a mouse.

93. The method of claim 90, wherein the subject is a human.

94. The method of claim 86, wherein the candidate mimetic agent is selected from the group consisting of a small molecule compound, a polypeptide, an intracellular antibody and a nucleic acid.

95. The method of claim 94, wherein the polypeptide comprises at least one modified amino acid residue.

96. The method of claim 94, wherein the intracellular antibody is a single chain antibody.

97. The method of claim 94, wherein the nucleic acid comprises a nucleic acid sequence encoding at least one nucleic acid selected from the group consisting of an antisense RNA and an interfering RNA.

98. The method of claim 94, wherein the nucleic acid comprises a nucleic acid selected from the group consisting of an antisense RNA and an interfering RNA.

99. The method of claim 94, wherein the nucleic acid comprises a plurality of nucleotides and wherein at least one of the plurality of nucleotides is modified.

100. The method of claim 86, wherein the exon 8 related loss-of-function modification is a mutation of an exon 8 related nucleic acid sequence selected from the group consisting of a deletion mutation, a frame shift mutation, a nonsense mutation, a missense mutation and a splice donor site mutation.

101. The method of claim 100, wherein the exon 8 related nucleic acid sequence comprises exon 8 of the WFS1 gene.

102. The method of claim 86, comprising determining the effect of the candidate mimic agent on WFS1-mediated apoptosis, wherein at least a partial inhibition of the WFS1-mediated apoptosis indicates that the candidate agent is a WFS1 mimetic.

103. The method of claim 86, comprising determining the effect of the candidate agent on WFS1-mediated calcium responses, wherein at least a partial restoration of the WFS1-mediated calcium responses indicates that the candidate agent is a WFS1 mimetic.

104. The method of claim 96, comprising determining the effect of the candidate agent on a WFS1-mediated phenotype selected from the group consisting of a muscle wasting disorders, denervation of diaphragms, respiratory arrhythmias and psychiatric disorders, wherein at least a partial inhibition or restoration of the WFS1-mediated phenotype indicates the candidate agent is a WFS1 mimetic.

105. A method for identifying a modulator of WFS1 in a motor neuron, comprising:
(a) contacting a motor neuron with a candidate agent; and
(b) determining an effect of the candidate agent on WFS1 function, wherein a change of the WFS1 function indicates that the candidate agent is a modulator of WFS1 in a motor neuron.

106. The method of claim 105, wherein the motor neuron is a spinal motor neuron.

107. The method of claim 105, wherein the motor neuron is in a subject.

108. The method of claim 107, wherein the subject is a rodent.

109. The method of claim 109, wherein the subject is a mouse.

110. The method of claim 107, wherein the subject is a human.

111. The method of claim 105, wherein the candidate agent is an agent selected from the group consisting of a small molecule compound, a polypeptide, an intracellular antibody and a nucleic acid.

112. The method of claim 111, wherein the polypeptide comprises at least one modified amino acid residue.

113. The method of claim 111, wherein the intracellular antibody is a single chain antibody.

114. The method of claim 111, wherein the nucleic acid comprises a nucleic acid sequence encoding at least one nucleic acid selected from the group consisting of an antisense RNA and an interfering RNA.

115. The method of claim 111, wherein the nucleic acid comprises a nucleic acid selected from the group consisting of an antisense RNA and an interfering RNA.

116. The method of claim 111, wherein the nucleic acid comprises a plurality of nucleotides and wherein at least one of the plurality of nucleotides is modified.

117. The method of claim 105, comprising determining the effect of the candidate agent on WFS1-mediated apoptosis, wherein a change of the WFS1-mediated apoptosis indicates that the candidate agent is a modulator of WFS1 in a motor neuron.

118. The method of claim 105, comprising determining the effect of the candidate agent on WFS1-mediated calcium responses, wherein a change of the WFS1-mediated calcium responses indicates that the candidate agent is a modulator of WFS1 in a motor neuron.

119. The method of claim 105, comprising determining the effect of the candidate agent on at least one WFS1-mediated phenotype selected from the group consisting of muscle wasting disorders, denervation of diaphragms, respiratory arrhythmias and psychiatric disorders, wherein at least a partial inhibition or restoration of the WFS1-mediated phenotype indicates the candidate agent is a modulator of WFS1 in a motor neuron.

120. A method for validating an animal model for a motor neuron associated disorder, comprising:
(a) determining a plurality of phenotypes of a candidate genetically modified animal under a given condition;
(b) determining a morphology of at least one motor neuron in the candidate genetically modified animal;
(c) optionally, determining a physiology of at least one motor neuron in the candidate generically modified animal;
(d) comparing the plurality of phenotypes with a plurality of phenotypes of an WFS1 model animal under the given condition, wherein the WFS1 model animal comprises a plurality of genetically modified somatic cells, wherein the plurality of genetically modified somatic cells comprises an exon 8 related loss-of-function modification in all WFS1 alleles; and
(e) comparing the morphology and, optionally, the physiology of the at least one motor neuron with the morphology and, optionally, of at least one motor neuron in the WFS1 model animal, wherein an essentially identical match between the plurality of phenotypes, the morphology and, optionally, the physiology indicates that the candidate animal is as a model animal for the motor neuron associated disorder.

121. The method of claim 120, wherein the motor neuron associated disorder is ALS.


SEQUENCE LISTING (5 sequences)

SEQ ID NO: 1 - DNA, 1193 bp, Homo sapiens
ataaaaaatgggctcagtgccacctccctccttgggtcactaggggggcccttgaaagctgtgaaagcccctaagccaacctggacagagcaggcgaggctgacggcaggtgttcttccttcctctgtgagcctcacagggtcacacgtgaacattggggatcgggggtgggcagtgggggcagctggagaatcaaatgaggtgactgacatgaactacgccgctttgacaaaagtcctccgaagtagtgtgttcagaactcatctccaaagccatctggaatatttgctcccaacatgttggtaagcattatctctgacagtcactccgagccccagtttctgctgtgataactaggacaggtagctcaggttggttgtggggaccagagacagagcaggcagggtctcacgttccccagggcctctcaaggatagaccctcgccctcatctccaaaccacgcctcccagacaggaaccaaactcccagagtctccaaactgcctgagccttgcccactccctgggctaacacacactttaaaggaatcccacagtcaccgtgtgaaaagcttgctacactgcatttgattctgggcactgaaagcagtacttggctgcagacactcgtttcaaacaggccccatttttccatctctgctgctgttattaggggagcccttagactctcttgcagcgccggaataggcgctcaagacgtgtgttaatattgcaacagcaaatataatgaatctgcagttggggacgctgaggccggagtggtggatgaaaggtggccggagccttttccacgggtccaaaccacctgttacaggagaaggcgagcggcctcgctaagcaactggacgttccgcgggcggggcgggggcggggccggggcccgagtccgctcggaaactttcgctgtgggcgagccggacccgccttctggcccctcgggcccaaccacgcagggggagtgccgggccgcagctaggcgaggcgcaccgtgattggcggagatgtggagtgattggcggctacaccggccactcagcaggccgagctggcgccgcatccggggggccgcgtctggagtggagggaggccgaaggccccgcccctgccccgccccctcgtgcagaaggccgcgctagccggctcttcagcagcgagtgcagattgctcccccgc

SEQ ID NO: 2 - DNA, 1554 bp, Homo sapiens
tttactattattaagcttattgttcttgctattatttactttcccccttaattttgcttccttactctccctccacccctcccccaacttctacctagtaattcaggagttctactctgcttctttcccgtgagttgctactttccaattcctgagagtcataattcaaccaatctttccctgataatgtattaaaagtaaatggtaatgaagccccctccccaccaccaataaggctttccttccccagctgagagtcataattaaaccaatctttccctgctaatgtattaaaagtaaatggtaatgaagcacccctcccaaccaccaataaggctttccttccccactcaccaaatcagatccttttacttcctccttctcccctcaaccaaatgtgatcttaaaatattctgacttctctgtgataagattctatttttttttttagttccaggctaatacaactttctcttgaagcatgtagcttatgttttaagaatccttatgtggcactgctagtacaaaatcacaggcacatgtggattatccatactgtatattttgcaagggtcattgcttaacaccacttcctgttctgagcgtttgctctatcttgaaatctctactgtgattgaactgtctcggtgacgggggaactctgaatatcttcctctgaaaggggacatcactgaggtcctttctctgtcttgccacagtggatattttctctcttttgtccacatagatgaatcctgtagtagccatggtaaggatccttgatcctatcatcagagtccctggacattgctccaaagtcccctaggaaccacttcaacttttctccccttagatgtgacctgttctttctggaactctgcaagaccgtctctttatccttggcatctgggatttccagggacacgttctcctggcactttgctcacctctgtttcccctccagctcttcctggagtacctgtcctttgtgcaatacacctcctggaactgtctttccagtctctgctttttgtcctgatgattttcatgtgcttttcctctacatcatgaagcatttactccacattatcttccagtttgctcatttgtcctcacctccactcttctctttctgaatttgcctgttgctttttcagtctcttagctccgtggagtatgttcagtgttgctgtgttctgatagaggctccttaagccatcttgtagattttttgttgcacttcctctcttcccttagacctccacaggaggaacttttcaatctgtcctcttctctctgcttaatgagcccctgcaaagggtttgggaagtgcttcagcctctctgtgattgtgtgttcacattaatatttattattttcttaaatgtgcataaatctcacaatgtgaacagtgatcatctttgagtgattttttttctcctttctacttttagtaattctccaaattttctacagttagtgtgtgataaggtggaaagacatcttcaagactttgggcaaataactatgtctgagcctcaattttcacatcaa

SEQ ID NO: 3 - DNA, 5802 bp, Homo sapiens
cggccgcagatctcccgtttgcgccgcgttcagctgctcccgaacaacttttctgccggcccagaggccccagggcgtcgcagcgccgcgtgcggcccactcacgggccggtgagtacttcggcgctggggcagtggcgcggtggctgtgggcagcgcgcaggaggcgggcaagagccctgaggcactgtcctcttcgggcctcagtttccccttccgagctgatgggtggctggccccaaagtcccgacaaggtccccgaagttggagggccgggggtcccgcccgctctgcaacgcgcaaggcgacccctgttccgggcccgaacgggtcacccggggggcgcgccccggtcccccgcgcgctgtcgcctggagccccgccgcgggcgggacagcaggcccgagaggcggagcggccggcgggcaggccccctccccgcgccccgcgccatcccccccgagttgccccgtcatgcccctcccccgcggtcccgggacccacggccgccctcggtgcccgcccggccgttggaacgcgcctgggcccccgggaggtgttgtgggggggaaggggggttcagtcccccagggggacccggcccgcccgaggggcagcttaggggtcattctgaatccccacagcccacttaggagcgctgtggcagttcctctctctctcggggcccctcagtagagagttgaggggtttcagtggcagcacctggaccctgcctcgtatgaactccaccctctggatcattagctttttgtttgatcaacgaaaactttctggagaatcctacccagcccttttggatgtgatggcgccctggcctctcacccacttagcagatgatgttatgaattccaaggaagtcgcatttgtgcaaccccccaccacccagatttaaccctctcagtagccttaggcggagaagttatagtccccgtttgacaccccaggaagctgaggctcagcagggtttggtgccagcccaggaccacgaagctgatggggcagagcctggtgtgaggcctctgccctatctctccagaagcagccccgtctctcctcaccttggacagtgacccctggcatttggcccagggctttgcagtttgtaaagggctttctcttatgccagcatccatcactcaaggagctgtgccctgggtaccccatgtgctggctgcctgttctctgcagcaggagagggagcccacgggaagcccagaggccacctttgtcactgtcgccacctgtttacttctgtgtccaagggtctgtgattcccatggggtctccgttgtatccaaccagggatttggagggaggcttctccacatttcagcctcttttccctacctgctggtttgtcctcccttcgcagacacaggaaaagttggctgggagaccccatctcaccttctttcccaggatggaagagcatggagggcgtctctgacttaggcctcccctgcccatcccggaccagtgctggacaggcttctggggctggtcacagaggggacctcagctcagtctcaacccccagagatgtctgcactgccagacagacccgaaacacccatccttgacggtggcaataagaatcataataggtcgggagctttatgcctacacggctcccctcccctcccacttccctacttccggaagtggtaccaccacagccagccagccccagccacaaaccagaccaccccctgtgcccttcattcctcactcccagttgggtctctcctggctcctgagcctcactcggatgcctgccctggttcaggaccctcgcctctccctggacgacccccaggctcctccctggtcccgatccgggcctggctgcacgcagccctgctggttcttcagctgccacttccctctgggctatcgggtcaggtccgcctcccgagccaggagccggcattctttcgggccctcagggacccgcctcgctgctcactcagcctctccctcctgtgtcctctctccagcagtcacctcccagccacctgcgtgttcctggccacaagcctggctcactgtccctctgctgcctcctcttctgggcacaacccctgctggaatgcccagccacctcccagcttgcctggctcctgcctgcttgtccagaaggctcagtcaagtcctcctgcaggatttctggcctgcctcttcccacagtggagtgagggcccccttctgtgccgtctttgtgggtgtctgtttaggtgttaacccttcccatctagatgaagctgctccaatgctggccctggacaagacttctctagggcaggggggcccagcacagtgcttggcacaagatggacctaccctcagtccacacctgctgcccatccaggccaggcctggagcaggtgccaggtgtgtggtgggggcttggccactatggccgtcatgaatgctgcttggggcagtcccaattcaaacccctctgttcctgtgaatgtgccacattggatagagctggaatgttccttggtttgtcccttaatactctgagcaaaaataggtacctgggatccatcaagggctttgtgcccttgtcttgaggagcagccctgtgccagggggctgggtcaggccaggccagagcagacatcccagggccaggacctcctgggcctgctcccttagagcttgctcacagatgggttttaaaataacatttgctttaatttcaaaaacatcacacattcattatagaaaactagggaaatatgtttaaaagaagaataaaatgaaaccgcgcataagctttatccccagagacaacaagtatcttttttagggggcgttttcttccgttttttggcagataaattttgttttcatcctaaacagactccggctaatatcttctgctgctttaaatcctgccttaacagtgttaggcccaccttttccttgtctcaaaacgttcatgtacgaggtcacgtttaccagctgtacagcgttgcaccatatggaggggtgtaggctttatagccagcccctttctggacagtgctgaaagtgccatatgatggacattgtgtgtgagccccgagggcatctccagtgactcccctggactggcatcctggaggcagaggcactgatcagagagagtgaacatcatcttggcttcccacaggggctgtttctgaggcactggcttgggagatggggttgcccccccagagagaacagaatgggggtggcctgggtgagggccagagcccttagggcagaaggccacgtgtggtcattctgctgcgggtctgagtggatgcatggtgcctgcagccctctccaggtggcggcctctcagctgaactccttggtgccggtgtttgtgtttggacgggataaaaatatgcctcattgctcatgaggaaagaatgctagcaactttggttttgtggatcttacacagtgacggcttctaagccattgctgtggtcaccagtagggagttggggctgagtgccccaccgtgcaggtggggatctgagggtgcagagtggatgcagctggagatgcctggatgctggggtctgggggagggacttgctaaggtcccacagcattattgcctggatggtgctcctgccctaggccaggctgcctcaggcttctgcccacatgcattaagtgcttaacgtattcctggcaaggcagccggtcttccgagtggataagctgcagccctgccctcagggctcacagtggctgtaacatgcaggaagactcagagagggaaggaccagacagcaggtccttccaccgggagatcttttctgatcttcattgtccctgtgaccaaccatgaccaggaattgttgtgcttgttttgaaataacaagccctttgatccaaaccagacttggccaacccccagggaacctgaaaattaatcctcctgtattcatccattcttactaaccagagattttactttaaacagtttttttttaactgcagtgtgacatacatggtacataggccagctagaatctgttttcctgaaattccttttatgaagtgttttccaactgaacatgcctttgtttagcacctgggagtaatggcttagggtttttcttttcctttttcttttttcttttttttgagacggagtctcgctttgtcacccaggctggagtgcagtggcgcgatctcggctcactgcaagctccatctcctgggttcacaccattctcctgcctcagcctccccagtagttgggactatcggcgcccgccaccacgcctggctaatttttttgtatttttaatagagacggggtttcactgtgttagccaggatggtcttgatctcctgaccttgtgatctgcccgcctcggcctcccaaagtgctgggattacaggcgtgagccaccgcgcctggccggcttagtttttttttaatgcccaatagtttgctttctcttctgccacacacgtaatccccatagcacccctatgtgggaggtgctatccccattttacatatggggaaacagacacagtgaggtcatgtggccactagtaagggtaaccgacgccccacaccatgttgcttccagctgtaacctctagcagagggagggctctgatggagaggtgatcgcctgagtgctgtgtgtttacctgagtaggtgaggaagacagggactcgggacagacatagcttggaagctcccacaaaggaggtatcgctggagctcttgacttgaagggtgcttagggtctaggctgggggaggggggtaagaaggcattccgggaagagggccctgcagaaacagaagtgtggaggtcagcatttgtatgctgatgttgaagggtagccagtgttttgcagtgaatgggccccgggagagagggtgcagtggcatggcggtctgatttcaaagtcgtctttttcagaaatgagcttgcagaagcattagaattgggcaggtctggatttgaatttcaggtctggtacctggagctgtttgaccttgagcaggtccctcccctccctgcacctgggttcctgtctgtgaagtggggtgaggttgaaggataaaacatttccccatctataactctgggatgacaataggatctaccttccaggtccatgcgaggaactcacgacaataggacctaccttccagggccatacgagaaactcatgagaaaagacaagtacagagaacagagccgttggtcttgatggcagagatgccctggcgtaggtcagccctcagtaaaggtgccctctctctctgtctgagaaagtgacacctgacttcggggcagtgggaaccatgggagagaaggtagacaaggccacgtgtcatttgaagacctgacttttcaccatctcaggtggtccggtggcctgcaggaccgtggttctgtttgcccacagcgtgtgggttctacgtgcgatcctgtatttctctgccacagctgactaaggggacacttaggtctgcacgtgcagttaaatggctgcttttgtggaaggtgccagttcccgggagctgggaacttggaaacaggatggagcaggctcccaagcttgaccatgcatggtttgcacctgggggatgcacccgggggtgcttgaccatgcatggtttgcacctgggggatgcacgggggtggggtggggggggggggtgtgcttgaccatgcatggtttgcacctgggggatgttttacactaagggctcagtccctctgagggaggaacgtgtggattgaaatctgcagag

SEQ ID NO: 4 - DNA, 961 bp, Homo sapiens
agtgacaaagtctggctttgtgacatgtgtgtttgtttcttctgtgttaaagggcctacaaagggagacatggaaatcccctttgaagaagtcctggagagggccaaggccggggaccccaaggcacagactgaggtgaggactgcggtgccggcagggacttcgggacgcggcccccggcacaacaggcctggccacgagctccacagcccacagagaagtgtcggtgcctgagatcggggtcaggagccagcgtggtgcaccctaccccacttgagccccatgttggtagggtgcccatgttcactgtgccagttttcctcctggcactcctctggggagcagcgctcatcccccttttgtccaactcacacctcatcttgggcatcacctcctccaggatgacctcctggcttcctgcagctgcctgctcagtgccacccctcaacatacactgtgtatgctgcccactcttgtctccctgactggcctttcacctggctgttagctgtgtgcacggggccctcagagcggtgaccattcacttgggcatcagccaagggctgggctttgtgccagtgctggggacatgatgtggccctctcttcatgggtgacaggttagtggaagaagcagacccaaaaaacccaagtagacaagacacagaaatacagtgattttgaattctggtgaggggagaagccagcagaacgctgagacgggaggtggcagggggtcctgtgtcctctgtggaggcaagttctcaccacctcactctgtccccttgcacagggctcaagcaagtgaccacagttatcctgagcagtttcctgccccctcttctccctaaagaggagaaaagcttccacgcgaaaccataaattaagaatttctggctgggagcggtggctcacgcctgtaatcccagcactttgggaggctgaggcgggtggatcacctg

SEQ ID NO: 5 - DNA, 1094 bp, Homo sapiens
cctgcagaagcagaggcgcatgctggagcgcctggtcagcagcgagtgtgagtgcagcccctgccccgtctcacccatgcctcccagcctgcacctgcagggcgacctctccttcctgtgcgactccatcctggcctgccctatctcacccgtgcctcccagcctgcgcctgcagggcgacctctccttcctgtgcgaccccatcctggccctgctaggatctcaggcggtccgtttggggctcagtgttctggacgctgggagtagaccctgcccacctggagcgcacgcactggagggaaggcagacccaggacagaaaccatgatgtgccagtccctcttggacaaggaaatactgggggtggggactggcgggggggtcctgagtggggaaggatgaggagggcattaggggaaggggctccgggcagagggaacagcatgagcaaaggcccaggagacccagcagggtgtggaaggggctctgccccagcgtaggccctttgtaggtaggcagggaacagagtgggcagagcctaggttgggggatagagtctctggggcacactgagctgggggtgcctctgggatgtttgtggtcaaagactgagaagagacgtcacatcctccacgtgcagctggccagcctcactgtcttccatccacgtggggtagactctgcagtctgaggcactgggagctcctgtcctggagaagatagacaggcaggaggcttgggggctcagtatcaatggggggatgggccaagacccaggtctgccacagactcttcttgtgacatgggcaggtctcatttttctttctgggtgtcagtttccccttctgaacagtgagatgatcagacaaggggactgaaggacctcgcccatcctagaagcctctgtggcatgtagggcagagtacaggtgggagcacagatcccaccttcccactgggcccgtgaggaaaccgtccatagggtgtggtcagagccctcgcctctgcgtgggcggacatgggtgcttgcgctgctgttttcctgctccatttgggggagacaaagaaacacctcacatggaagtgggaagaaatc